(19) United States
(12) Patent Application Publication
(10) Pub. No.: US 2006/0188398 A1
(43) Pub. Date: Aug. 24, 2006

(54) Sensor for detecting a target substance in a fluid

(75) Inventors: Koji Yano, Tokyo (JP); Takeshi Imamura, Tokyo (JP); Mitsuro Sugita, Tokyo (JP); Kohei Okamoto, Kanagawa-ken (JP); Takeo Yamazaki, Kanagawa-ken (JP); Hidenori Shiotsuka, Kanagawa-ken (JP); Toshihiko Ouchi, Kanagawa-ken (JP); Ryo Kuroda, Kanagawa-ken (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 10/553,977

(22) PCT Filed: Aug. 27, 2004
(86) PCT No.: PCT/JP04/12790

§ 371 Date: Oct. 20, 2005

(30) Foreign Application Priority Data
Aug. 27, 2003 (JP) 2003-303115
Aug. 27, 2003 (JP) 2003-302520
Aug. 26, 2004 (JP) 2004-247468

(51) Int. Cl. G01N 33/50

(52) U.S. Cl. 422/82.01

(57) ABSTRACT

A device for detecting a target substance in a fluid is provided. This device comprises a periodic structure having a vacant portion for passing the fluid containing the target substance and a solid portion arranged regularly and capable of transmitting an electromagnetic wave, an electro-magnetic wave-projecting means for projecting the electromagnetic wave to the periodic structure, and a detecting means for measuring the magnetic wave emitted from the periodic structure to detect a change in periodic distribution of a refractive index. This sensor is highly sensitive in a small size.

### TECHNICAL FIELD

The present invention relates to a sensor technique for detecting a pollutant in the air, a specific substance in the blood, and the like substances.

### BACKGROUND ART

With growing importance of inspection technique for biological substances such as blood, various sensing methods are being developed in recent years. Many methods for detection of a trace amount of pollutants have been disclosed. The common problem of these methods is to detect a specified object substance in a liquid or a gas.

One method for the above detection is disclosed in which an antibody is deposited on a photonic crystal and the antibody-antigen complex formation is detected by change in the spectrum of the reflected light from the photonic crystal (Adv. Mater. 2002, 14, No.22, p. 1629 (2002)).

### DISCLOSURE OF THE INVENTION

According to an aspect of the present invention, there is provided a device for detecting a target substance in a fluid, comprising

a periodic structure having a vacant portion for passing a fluid containing the target substance and a solid portion capable of transmitting an electromagnetic wave arranged regularly to form a periodic distribution of a refractive index for the electromagnetic wave,

an electromagnetic wave-projecting means for projecting the electromagnetic wave to the periodic structure, and

a detecting means for measuring the magnetic wave emitted from the periodic structure to detect a change in the periodic distribution of the refractive index.

According to another aspect of the present invention, there is provided a device for detecting a target substance in a fluid, comprising

a flow path for passing a fluid containing the target substance,

a periodic structure placed at least a portion of the flow path and having a vacant portion for passing the fluid containing the target substance and a solid portion capable of transmitting an electromagnetic wave arranged regularly to form a periodic distribution of a refractive index for the electromagnetic wave,

an electromagnetic wave-projecting means for projecting the electromagnetic wave to the periodic structure, and

a detecting means for measuring the magnetic wave emitted from the periodic structure to detect a change in the periodic distribution of a refractive index.

According to a further aspect of the present invention, there is provided a device for detecting plural target substances in a fluid, comprising

a flow path for passing a fluid containing the target substances;

plural periodic structures each of which is placed at least a portion of the flow path and has a vacant portion for passing the fluid containing the target substances and a solid portion capable of transmitting an electromagnetic wave arranged regularly to form a periodic distribution of a refractive index for the electromagnetic wave, an electromagnetic wave-projecting means for projecting the electromagnetic wave to the periodic structures, and

a detecting means for measuring the magnetic wave emitted from the periodic structures to detect a change in the periodic distribution of the refractive index.

According to a further aspect of the present invention, there is provided a device for detecting a target substance in a fluid, comprising

an optical fiber having plural holes for passing the fluid containing the target substance and a solid portion capable of transmitting an electromagnetic wave to form a refractive index distribution in the radius direction,

an electromagnetic wave-introducing means for introducing the electromagnetic wave to the optical fiber, and

a detecting means for measuring the magnetic wave emitted from the optical fiber in the radius direction to detect a change in a refractive index.

### BRIEF DESCRIPTION OF THE DRAWINGS

FIG. 1 illustrates an example of a band structure.

FIG. 2 is a graph showing transmittance.

FIG. 3 illustrates a photonic crystal structure.

FIG. 4 illustrates a state of a trapping substance and a photonic crystal.

FIGS. 5A and 5B illustrate a state of the surface of a trapping substance and a photonic crystalline substance when a test sample liquid is allowed to flow.

FIG. 6 is a drawing illustrating a one-dimensional periodic structure.

FIG. 7 is a drawing illustrating a three-dimensional periodic structure.

FIG. 8 is a drawing illustrating a two-dimensional periodic structure.

FIG. 9A is a perspective view of a two-dimensional photonic crystal having periodic holes formed therein.

FIG. 9B is a sectional view of the photonic crystal shown in FIG. 9A.

FIG. 10 is illustrates the trapping substance applied to the photonic crystal substance.

FIG. 11 illustrates detection of transmitted light.

FIG. 12 illustrates a photonic crystal structure having a defect.

FIG. 13 illustrates another photonic crystal structure having a defect.

FIG. 14 illustrates still another photonic crystal structure having a defect.

FIG. 15 illustrates introduction of light into a photonic crystal having a defect.

FIG. 16 shows a spectrum of light having been transmitted through a photonic crystal having a defect.

FIG. 17 illustrates a state of a photonic crystal having a defect and a trapping substance deposited thereon.

FIG. 18 illustrates detection of a target substance in a photonic crystal having a defect.

FIG. 19A shows change in transmission spectrum by trapping of a target substance by a trapping substance around resonance.

FIG. 19B shows change in transmission spectrum by trapping of a target substance by a trapping substance around resonance.

FIG. 20 shows a method for detection of change in a path of introduced light.

FIG. 21 shows a path of light through a circular emission face.

FIG. 22 shows propagation of optical energy at the photonic crystal interface.

FIG. 23 shows propagation of optical energy at the photonic crystal interface when a superprism effect is produced.

FIG. 24A is a perspective view of the constitution employed in Embodiment A-1.

FIG. 24B is a plan view of the constitution of FIG. 24A.

FIG. 24C is a sectional view of the constitution of FIG. 24B.

FIG. 25 illustrates the constitution of the sensor employed in Embodiment A-1.

FIG. 26 illustrates the constitution of the sensor employed in Embodiment A-2.

FIG. 27 illustrates the constitution of the sensor employed in Embodiment A-3.

FIG. 28 illustrates the constitution employed in Embodiment C-1.

FIG. 29 illustrates the constitution of the sensor employed in Embodiment C-1.

FIG. 30 illustrates the constitution of the sensor employed in Embodiment C-2.

FIG. 31 illustrates the constitution of the sensor employed in Embodiment D.

FIG. 32 illustrates the constitution of the sensor employed in Embodiment D.

FIGS. 33A and 33B illustrate the constitution of the sensor employed in Embodiment E.

FIG. 34 illustrates a constitution of a photonic crystal fiber.

FIG. 35 illustrates a constitution of a sensor employing a photonic crystal fiber.

FIG. 36 illustrates another constitution of a photonic crystal fiber.

FIG. 37A illustrates a constitution of another sensor employing a photonic crystal fiber.

FIG. 37B is a partial sectional view of the constitution of FIG. 37A.

FIG. 38 illustrates the constitution of the biosensor employed in Example 1.

FIG. 39 illustrates the constitution of the biosensor employed in Example 2.

FIG. 40 illustrates the constitution of the sensor employed in Example 3.

FIG. 41 is a sectional view illustrating the constitution of the sensor employed in Example 3.

FIG. 42 illustrates the constitution of the sensor employed in Example 3.

FIG. 43 is a sectional view illustrating the constitution of the sensor employed in Example 3.

FIG. 44 illustrates the constitution of the sensor employed in Example 4.

FIG. 45 is a sectional view illustrating the constitution of the sensor employed in Example 4.

FIG. 46 illustrates the constitution of the sensor employed in Example 4.

FIG. 47 is a sectional view illustrating the constitution of the sensor employed in Example 4.

FIG. 48 illustrates the constitution of the sensor employed in Example 5.

FIG. 49 is a sectional view illustrating the constitution of the sensor employed in Example 5.

FIG. 50 illustrates the constitution of the sensor employed in Example 5.

FIG. 51 is a sectional view illustrating the constitution of the sensor employed in Example 5.

FIG. 52 illustrates the constitution of the sensor employed in Example 6.

FIG. 53 is a sectional view illustrating the constitution of the sensor employed in Example 6.

### BEST MODE FOR CARRYING OUT THE INVENTION

1. Photonic Crystal

The sensor of the present invention employs a photonic crystal for detecting a target substance. Generally, the photonic crystal is a periodic structure having periodic refractivity distribution for light (or electromagnetic wave). The periodic structure can be formed by arranging periodically substances having different refractivities, or by forming a periodic structure from one single substance having aerial or vacuum gaps therein. The periodic structure may be one-dimensional, two-dimensional, or three-dimensional. The cyclic period corresponds nearly to the wavelength of the electromagnetic wave to be treated. For example, for treating a light beam of a wavelength of 800 nm, the cyclic period of the periodic structure may be 200 nm or 400 nm.

The photonic crystal has a photonic band structure depending on the crystal structure and the energy and wavelength of the light. The remarkable feature is formation of a photonic band gap by design of a periodic structure in which a certain wavelength band cannot exist in this photonic crystal structure. The light of this wavelength band cannot propagate through the photonic crystal. Therefore, this light irradiated from outside cannot enter the photonic crystal and is reflected. Further, the photonic crystal having portions of a different refractivity cycle period is known to show an interesting phenomenon such that the light is allowed to travel through the limited path or is confined inside the crystal without emitting outside.

The periodic change in the refractive index or periodic structure causes change in the photonic band gap. The photonic band gap depends on the light travel direction and the polarization plane.

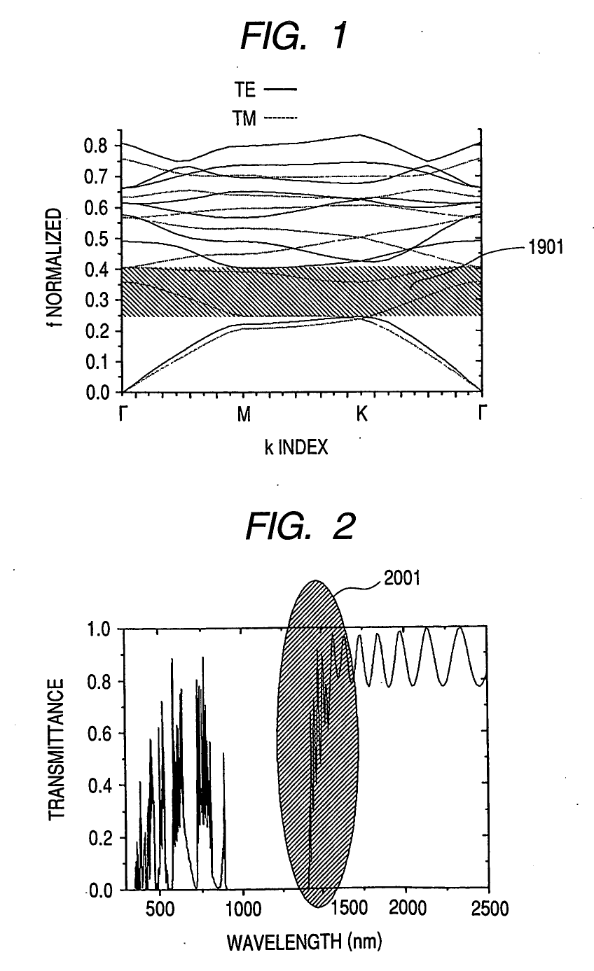

FIG. 1 shows the result of calculation of the band structure formed in silicon for the case where a periodic structure has the lattice constant “a” and has holes of the radius of 0.4a arranged periodically in two-dimensional direction. The abscissa indicates the wave vector of the electromagnetic wave in the two-dimensional plane of the periodic structure, and the ordinate indicates the normalized frequency of the electromagnetic wave.

In the two-dimensional periodic structure, the mode of the polarized electromagnetic wave perpendicular to the plane is called a TE mode, and the one parallel to the plane is called a TM mode. In FIG. 1, the dotted lines show the TM mode, and the solid lines show the TE mode. Noticing the TE mode, there is a region 1901, a band gap, where no electromagnetic wave can exist regardless of the wave vector. In the frequency axis, the both ends of the band gap are called band edges.

FIG. 2 shows the calculation results of the transmittance of the electromagnetic wave as a function of the wavelength of the electromagnetic wave at the wave vector of K, assuming the lattice constant “a” to be 350 nm. A band gap in which the transmittance is approximately zero is found in the wavelength range from about 900 nm to 1400 nm.

The above characteristics can be shown for lights and electromagnetic wave other than the visible light such as infrared light and ultraviolet light. However, the electromagnetic wave is selected preferably from visible range or near thereto, since the sensor device is preferably smaller in size.

2. Detection Principle

The photonic crystal employed in the present invention is constituted of solid portions formed from a light-transmissive material and empty portion (also called a vacant structure) containing no substance. The solid portion is formed from a dielectric material like silicon, and the vacant structure is exemplified by pores formed in the silicon. The periodic structure is a one-, two-, or three-dimensional periodic arrangement of the vacant structure and the solid portions. A trapping substance capable of bonding to the target substance is placed preliminarily in the photonic crystal.

On introduction of a fluid containing the target substance into the empty portion, the target substance in the fluid causes a bonding reaction with the trapping substance supported on the surface of the solid portion of the periodic structure during the flow of the target substance-containing fluid through the empty portion.

The bonding reaction of the target substance and the trapping substance changes refractive index of the surface of the solid portion, which changes the photonic band structure thereof. For instance, in FIG. 2, the band edge 2001 is changed or shifted by the bonding reaction. Therefore, the target substance can be detected by detecting this change. The change in the photonic band structure can be detected by change in the intensity of the transmitted light or reflected light, or change in the light traveling direction. The method of the detection is described later specifically.

As described above, the target substance can be detected by depositing preliminarily a trapping substance capable of bonding selectively to the target substance to be detected.

The sensor device employing the photonic crystal is necessarily comprises a flow path, a photonic crystal placed in a portion of the flow path, a projecting means for projecting an electromagnetic wave to the photonic crystal, and a detecting means for detecting the change in the photonic band structure by measuring the electromagnetic wave emitted from the photonic crystal.

The material of the photonic crystal includes various substances such as semiconductors like silicon, and gallium-arsine; glass; and resins.

The wavelength band range of the electromagnetic wave for detection is selected to be most suitable in constitution of the actual sensor, and may be of a single wavelength or a part of a wavelength range having a certain breadth.

The periodic structure is placed at least in a part of the flow path of the fluid, but may be filled in the entire of the flow path. Further, the periodic structure need not be entirely included in the flow path, but may be partly placed outside the flow path.

The electromagnetic wave-projecting means comprises an electromagnetic wave source. The electromagnetic wave source is exemplified by a laser. The light beam, an electromagnetic wave, emitted from the laser is collimated by a lens or the like to be projected for the sensing. Thus the electromagnetic wave-projecting means comprises all of the necessary elements for generating and emitting an electromagnetic wave for the sensing operation.

The sensor comprises an electromagnetic wave detector for detecting the signal electromagnetic wave. The electromagnetic wave is projected by the electromagnetic wave-projecting means to the periodic structure. The signal electromagnetic wave transmitted through the periodic structure or reflected thereby is detected by the electromagnetic detector. The electromagnetic wave detector is exemplified by a photodiode, and a CCD (charge coupled device) detector. The presence of the target substance is detected by the change in the detection results before and after the sensing. As mentioned above, the target substance of the detection object is detected with such constitution of the sensor of the present invention.

When a fluid containing the target substance is allowed to flow through the vacant structure of the periodic structure, the band structure of the periodic structure becomes different from the band structure in a state of the vacant structure filled with a fluid not containing the target substance.

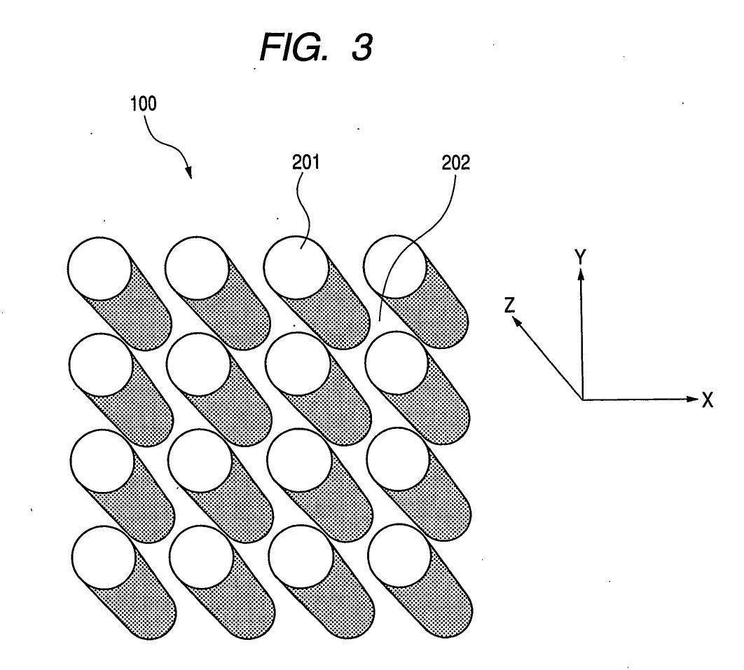

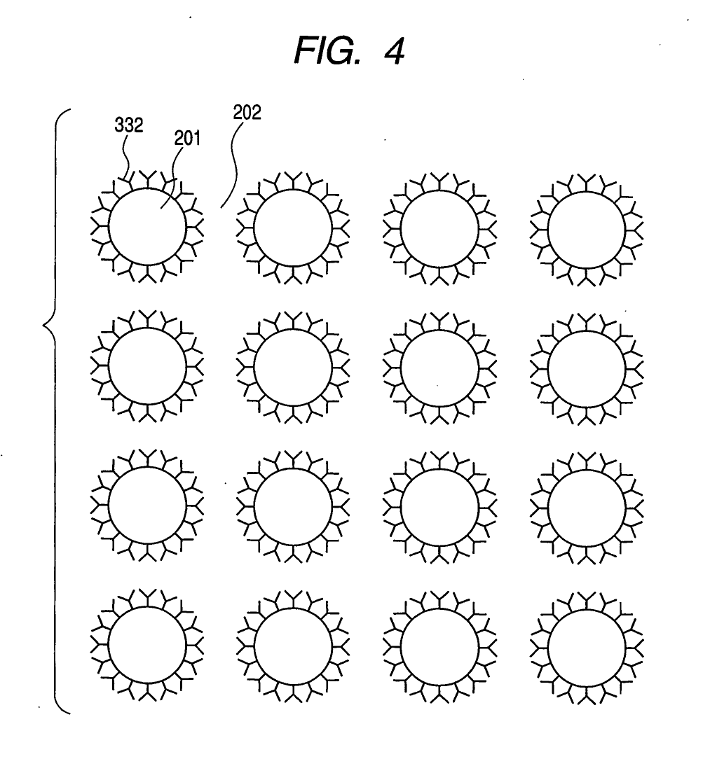

FIG. 3 illustrates an example of the photonic crystal employed in the present invention. The coordinates X, Y, and Z in FIG. 3 and other drawings are shown for convenience of explanation.

Photonic crystal 100 in FIG. 3 is a two-dimensional photonic crystal constituted of constructing members 201. In FIG. 3, constructing members 201 are in a shape of a column directing in the Z-axis direction, and are arranged regularly on the XY plane. (Hereinafter, the plane of regular arrangement in the two or more-dimensional photonic crystal is called a “two-dimensional plane”.) Although not shown in the drawing, these columnar members 201 are fixed on a suitable substrate to keep the cycle period. Incidentally, the directions of the coordinates X, Y, and Z in other drawings employing a two-dimensional photonic crystal are the same as the regular arrangement directions in FIG. 3. Therefore, the plane parallel to the two-dimensional plane is defined as the XY plane. The material of the constructing members is not limited insofar as it is transparent to the employed light, and includes semiconductors such as silicon, and gallium-arsine; resins; and SiO₂.

Vacant structure 202 signifies the portion not occupied by the constructing member. A fluid containing a target substance is allowed to flow through the vacant portion. (Hereinafter, the fluid is called a test sample fluid, or a test sample liquid. However, the object of the detection with the sensor of the present invention is not limited to the liquid.) Constructing members 201 and vacant structure 202 form a two-dimensional periodic structure. The cycle periods in the respective dimensional directions may be independent from each other. In the present invention, a photonic crystal is employed which has a photonic band gap in the light introduction direction for the detection.

The cycle period length is approximately the wavelength of the detecting light or shorter, ranging from 100 nm to 10 μm. The shape of the constructing member is not limited to be columnar, but may have an arbitrary sectional shape such as a rectangle and an ellipsoid.

FIG. 4 is a sectional view of the photonic crystal of FIG. 3 along the XY plane, illustrating a state of a trapping substance as the sensor substance disposed in a photonic crystal. Trapping substance 332 for trapping the detection object is adhering onto the surface of constructing member 201 facing to vacant structure 202. The detection sensitivity can be changed by varying the adhesion density of the trapping substance.

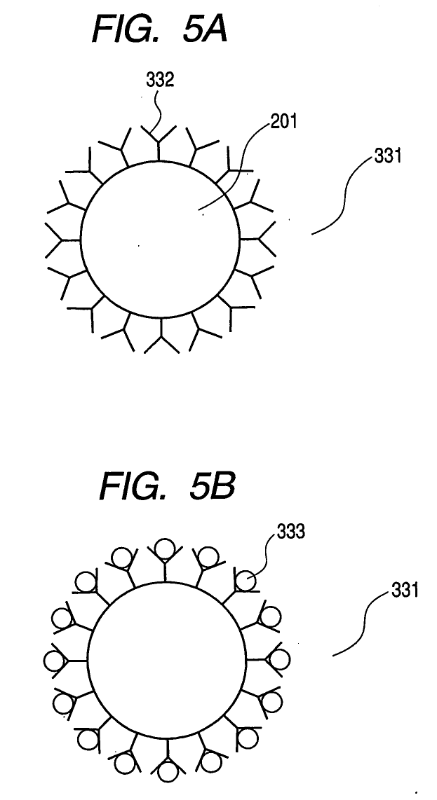

FIGS. 5A and 5B shows the change in the surface of constructing member 201 by a flow of test sample liquid 331. FIG. 5A illustrates the result with the test sample liquid not containing target substance 333, and FIG. 5B illustrates the result with the test sample liquid containing target substance 333. With a test sample liquid not containing target substance 333, trapping substance 332 is kept unchanged, whereas with a test sample liquid containing the target substance, the trapping substance 332 traps the target substance to come into a trapping state.

The bonding reaction is considered to occur immediately when the target substance and the trapping substance are brought close together. However, the bonding reaction of plural trapping substances distributed in a low density or high density of one target substance with one trapping substance on the solid surface with plural target substances dispersed in the fluid will proceed slowly as a whole, and will take a certain time for completion of the bonding reaction.

The sensor of the present invention detects the presence, kind, or concentration of the target substance by measuring the change in the periodic structure characteristics or the band structure caused by the bonding reaction by utilizing an electromagnetic wave. The measurement is conducted at a specified time after start of the flow of the test sample fluid through the periodic structure supporting the trapping substance. The specified time herein signifies a time necessary for the bonding reaction sufficient for measurement, by utilizing an electromagnetic wave, of the change in the periodic structure characteristics or of the band structure from the time of start of the flow of the test sample fluid through the periodic structure. The specified time depends on the kinds and materials of the trapping substance, the target substance, the fluid, and periodic structure.

During the flow of the test sample liquid, vacant structure 202 is filled with test sample liquid 331. When the test sample liquid does not contain the target substance, nothing is trapped by the trapping substance 332 during the flow not to cause change in the refractive index around constructing member 201. On the other hand, when test sample liquid 331 contains target substance 333, the region of trapping substance 332 comes to be in a trapping state as shown FIG. 5B, resulting in change in the refractive index by the trapping.

The photonic band structure of photonic crystal 100 is modulated in correspondence with the refractive index change. This modulation shifts the edge of the photonic band gap to cause change in the light transmission intensity, light reflection intensity, light propagation route in the crystal, and so forth of the light employed. By measurement of the change, the target substance contained in the test sample liquid is detected, and the quantity thereof is determined.

This measurement may be conducted with the test sample liquid filled in the photonic crystal structure. Otherwise, the measurement may be conducted after removal of the test sample liquid. The photonic band structure depends on the presence or absence of the test sample liquid. Therefore, when the measurement is conducted after removal of the test sample liquid, the presence of the target substance can be detected by comparison of the measurement data of the detection-treated periodic structure with the measurement data of another periodic structure having no target substance without the test sample liquid.

When the test sample liquid is replaced by another one, the measurement is conducted in the same manner.

The trapping substance may be disposed throughout the entire region or in a limited region of the photonic crystal. Otherwise the test sample liquid is allowed to flow in a limited region thereof, and the change in the photonic band structure is measured by light projection onto the limited region.

3. Dimension of Photonic Crystal

The present invention is applicable regardless of the numerical dimension of the photonic crystal.

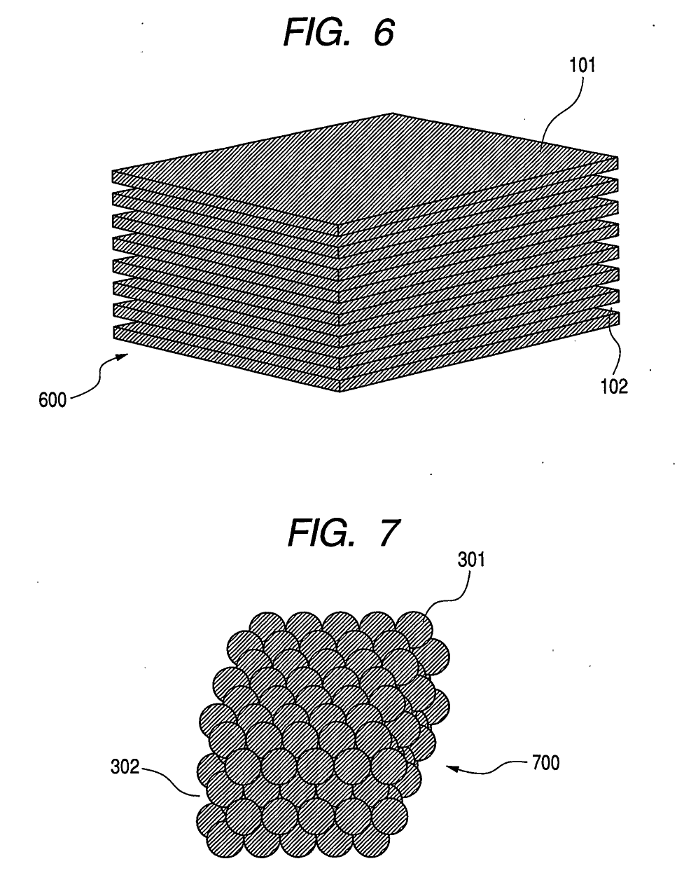

An example of the one-dimensional periodic structure 600 is shown in FIG. 6. In this structure, solid parts 101, thin films of a high refractive-index material, are placed periodically with interposition of gaps 102, a vacant structure.

An example of the three-dimensional periodic structure 700 is shown in FIG. 7. In this structure, fine spheres 301 as the solid parts of the present invention are arranged three-dimensionally in a hexagonal close-packed state, and the interstices 302 of solid portion 301 serve as the empty portion.

The three-dimensional photonic crystal is advantageous in that the freedom degree in designing the photonic band structure is high, but has disadvantageous in that the flow path is not straight in any liquid flow direction owing to the three-dimensional structure, being liable to cause stagnation or irregularity of the flow. This causes irregularity of the flow rate of the test sample liquid to cause nonuniform adhesion of the target substance, tending to lower the detection sensitivity or lower the reliability of the detection results.

Use of the two-dimensional photonic crystal is advantageous in that the stagnation or spatial irregularity of the flow is not caused.

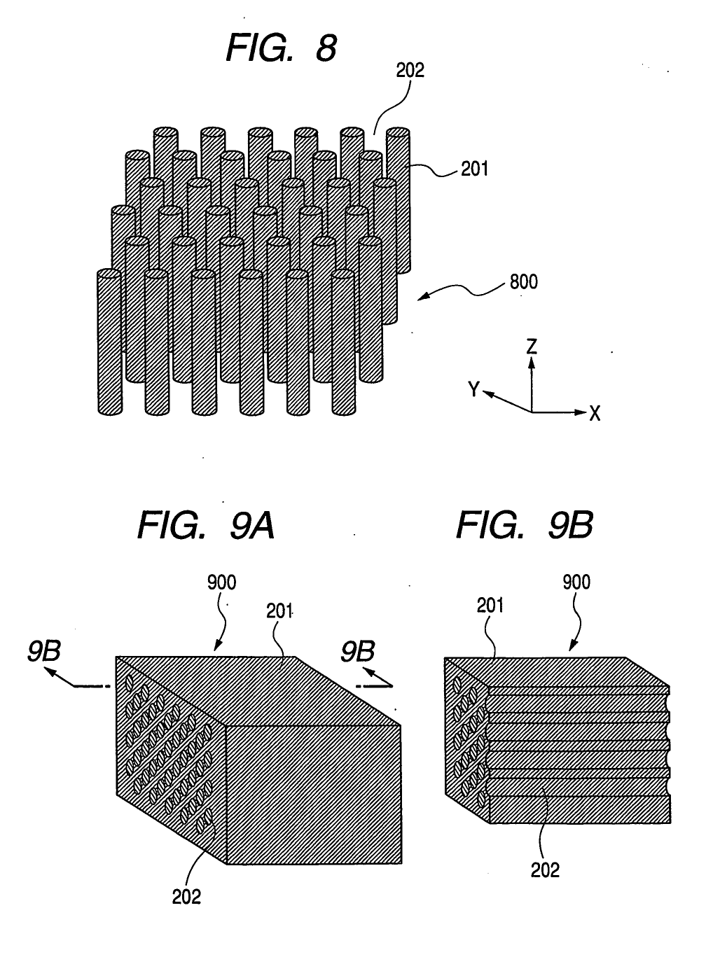

FIG. 8 shows a two-dimensional periodic structure 800 different from the one shown in FIG. 3 having the solid members arranged in a square lattice. The periodic structure shown in FIG. 8 has silicon columns 201 of 1 μm in height and about 110 nm in radius as solid members arranged two-dimensionally in a triangular lattice of a lattice constant of about 390 nm, and the interstices 403 serve as the vacant structure. The entire size of the periodic structure is about 100 μm in length and about 100 μm in width.

FIGS. 9A and 9B shows still another two-dimensional periodic structure 900. FIG. 9B shows a section at 9B-9B in FIG. 9A. In this structure, the solid member 201 is a continuous body, and therein holes 202, empty portions, are arranged periodically.

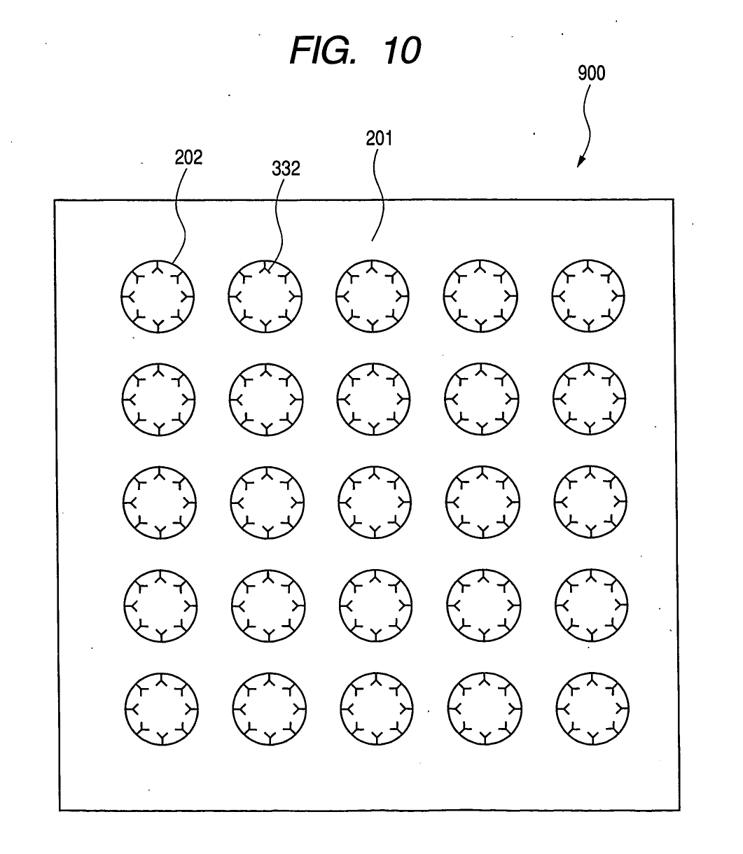

In such a photonic crystal having holes formed in the structure material, the trapping substance 332 to react with the target substance is allowed to adhere on the wall faces of holes 202 of structure 201 as shown in FIG. 10. The test sample liquid is allowed to flow through the holes shown in FIGS. 9A and 9B. The direction of the length of the hole is called herein the “axis direction”. Since there is no irregularity in the axis direction of flow of the test sample liquid, the test sample liquid passes smoothly through the holes without stagnation, causing bond formation of the target substance with the trapping substance uniformly.

In this embodiment, the fluid containing the target substance can be made to flow in a specified direction in correspondence with the photonic crystal structure. By selecting the placement of the photonic crystal to be suitable for the detection light irradiation direction, the measurement of the electromagnetic wave emitted from the periodic structure as well as the detection of the change in the periodic distribution of the refractive index can be conducted concurrently. Plural sensors may be placed in one flow path. In consideration of the advantages of the present invention, two-dimensional photonic crystals are preferred as the photonic crystal in the present invention.

4. Method of Detection

The change in the photonic band structure by adhesion of a detection target substance to a trapping material can be detected by any of the methods (1) to (3).

(1) Change in the photonic band gap is detected by measurement of light transmittance or reflectivity, or change in the photonic band gap edge is detected by measurement of transmitted light spectrum by use of a wavelength-variable light source at or around the wavelength of the band gap edge;

(2) A structure defect is formed to disorder the periodicity of the refractive index in the photonic crystal to form a defective level in the band gap, and the change in the transmitted light intensity is observed; and

(3) Change in the path way of light traveling through the photonic crystal structure is detected which is caused by change in the photonic band structure.

The methods are described below in detail.

(Detection Method 1)

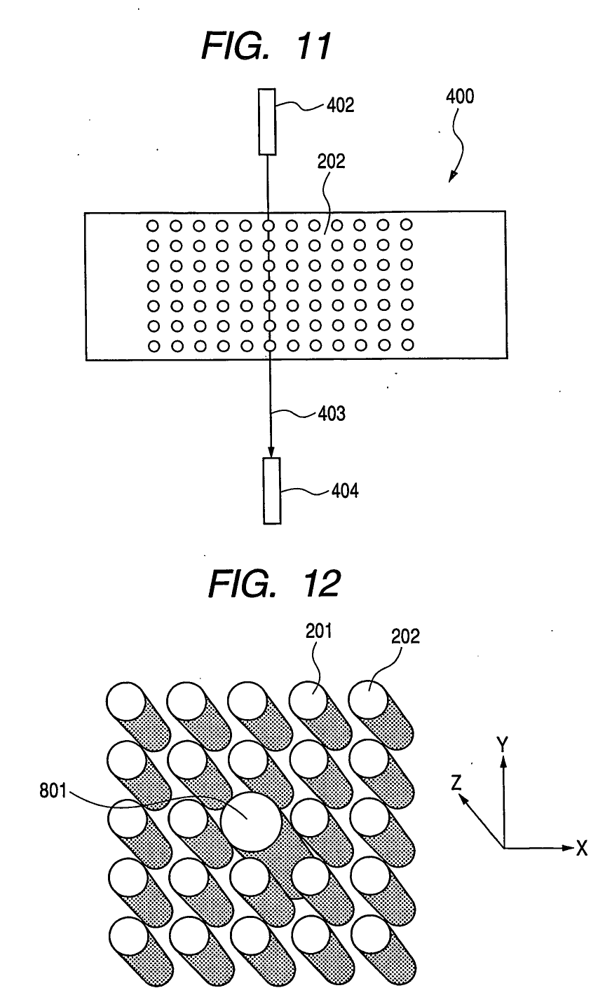

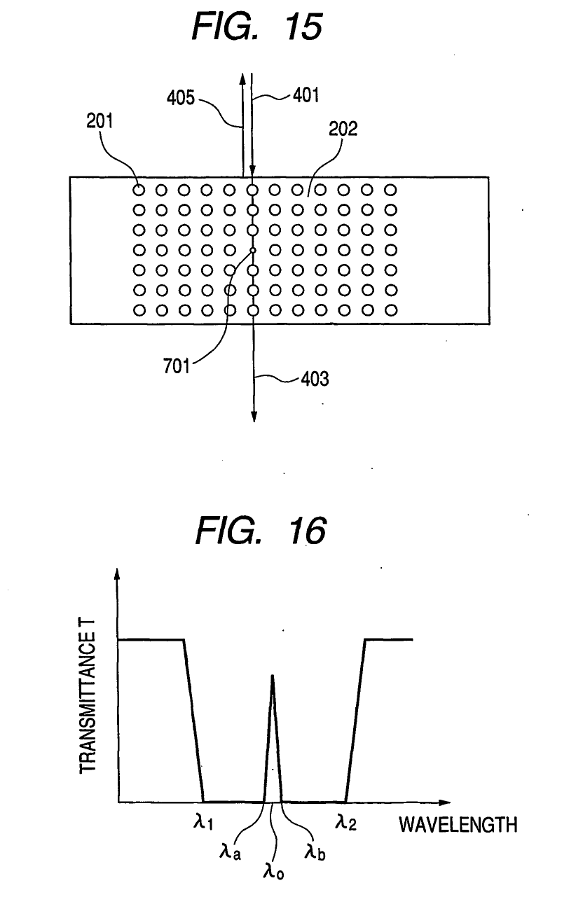

In FIG. 11, the numeral 400 indicates a photonic crystal, the numeral 402 indicates, a light projecting means for projecting light such as laser. Photonic crystal 400 employed has columnar structural members arranged regularly as shown in FIG. 3. In FIG. 11, the two-dimensional plane of the photonic crystal is indicated schematically by a matrix of blank circles. This indication is the same in FIGS. 15, 18, 20, and 21. A test sample liquid is passed through the gaps of the columns to trap the target substance. Transmitted light 403 is detected by signal light detector 404. Reflected light may be detected instead of detection of the transmitted light.

Light source 402 emits light of wavelength at or around the photonic band gap edge, and detector 404 detects the intensity change in the light at the same wavelength.

For the detection, light of wavelength inside the photonic band gap at the long wavelength edge is projected and the transmitted light is observed. When the band gap is shifted to a shorter wavelength side, the wavelength of the projected light comes out of the shifted band gap. Therefore the shift is observed as increase of the transmitted light intensity. The wavelength of the light to be projected to the sample is selected in the vicinity to the band edge. Naturally, the vicinity signifies the range of the shift of the band edge by trapping of the target substance.

Otherwise, a wavelength-variable laser is employed as light source 402, and the wavelength is scanned in the range including the long wavelength edge or the short wavelength edge to measure the change in the transmitted light spectrum.

(Detection Method 2)

FIG. 12 illustrates a photonic crystal of a columnar structure having a defect. Defective columnar structure member 801 is thicker in comparison with adjacent columnar structure members 201.

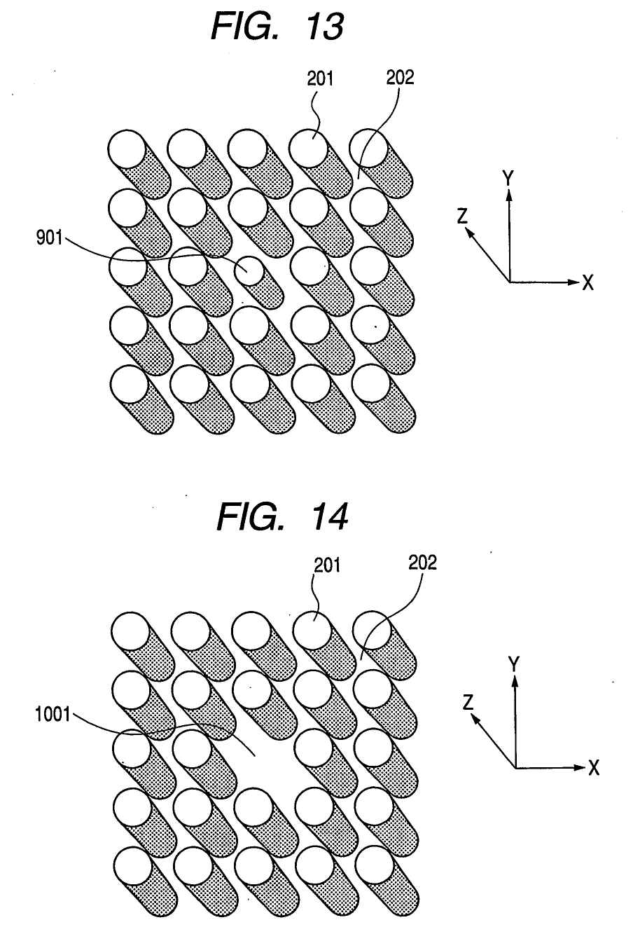

The defect may be columnar structure 901 having a smaller diameter than the adjacent columnar structure member as shown in FIG. 13, or lack 1001 of the columnar structure member as shown in FIG. 14. The type of the defect is not limited insofar as it disorders the periodic structure. However, for the detection in the present invention, the level formed by the crystal defect should be within the photonic band gap.

As shown in FIG. 15, light beam 401 is projected to a photonic crystal having a defective structure to shoot at the defective site 701. In FIG. 15, the numeral 403 denotes transmitted light and the numeral 405 denotes reflected light.

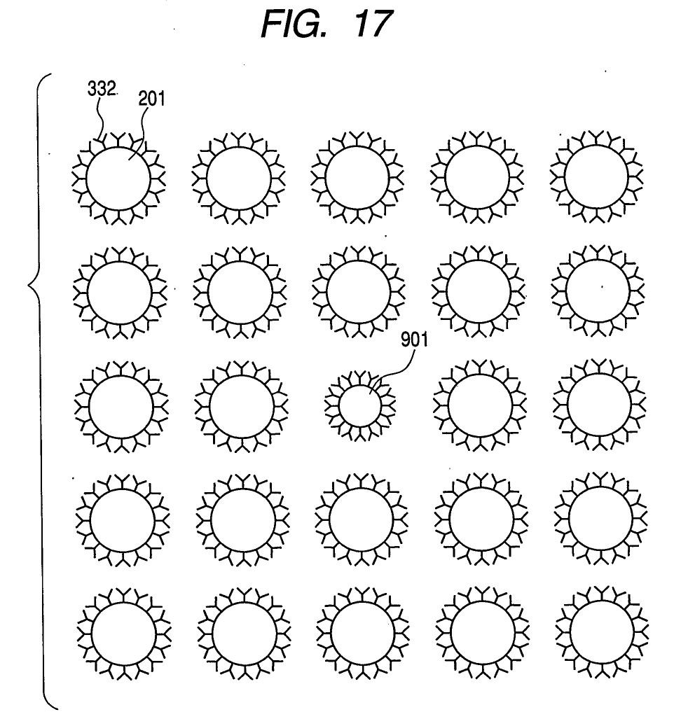

FIG. 16 shows schematically the transmitted light spectrum: the abscissa indicates a wavelength, and the ordinate indicates a light transmittance. The wavelength range between λ₁ and λ₂ is the photonic band gap. Within the photonic band gap, a resonance level λ₀ is produced by the defect, allowing the light of the wavelengths around λ₀ between λₐ to λ_{b} to pass through.

The breadth and height of the transmittance wavelength band in the band gap depend on the thickness of the photonic crystal along the light path: at the smaller thickness, the transmittance approaches 1 but the resonance is weakened to expand the width between λₐ and λ_{b}.

FIG. 16 shows the spectrum obtained by projecting the light beam to shoot at the defect, and the transmittance wavelength band does not appear in the band gap region except the defective site. Since spread of the projected light beam decreases the transmittance at this transmittance wavelength band to make difficult the observation of the change, the projected light should be sufficiently collimated and focused.

FIG. 17 is a sectional view of the photonic crystal shown in FIG. 13 at an XY plane, illustrating the state of the trapping substance and the photonic crystal. On the surface of constructing member 201 including a defective member 901, trapping substance 332 capable of bonding to a target substance to be detected is allowed to adhere.

When the target substance is trapped, the refractive index of the portion trapping the target substance is changed by the trapping. This change occurs similarly at the site of the defective member 901. This modulates the photonic band structure of the photonic crystal, and changes also the energy level of the defect. Thus the respective wavelengths shown in FIG. 16 are shifted. The sensor of the present invention detects the target substance by detecting the change in the light transmittance or reflective index caused by the defect energy level.

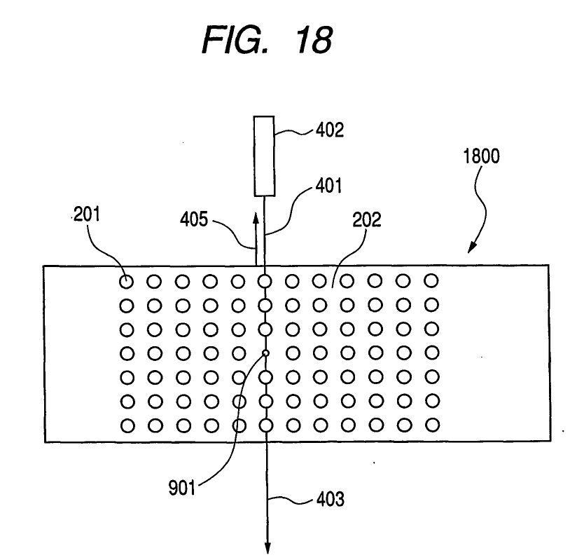

FIG. 18 shows a method of detection of a target substance according to the present invention. Photonic crystal structure 1800 has a defect 901 as described above, and contains trapping substance 332 as shown in FIG. 17. A light beam is projected from light source 402 to shoot at defect 901 of the photonic crystal. Into vacant structure 202 in the photonic crystal structure, a test sample liquid to be inspected is introduced. The target substance contained in the test sample liquid is trapped by the trapping substance, causing change in the transmission spectrum or reflection spectrum. In the present invention, in particular, is detected the changes in transmitted light and reflected light caused by resonance caused by the defect.

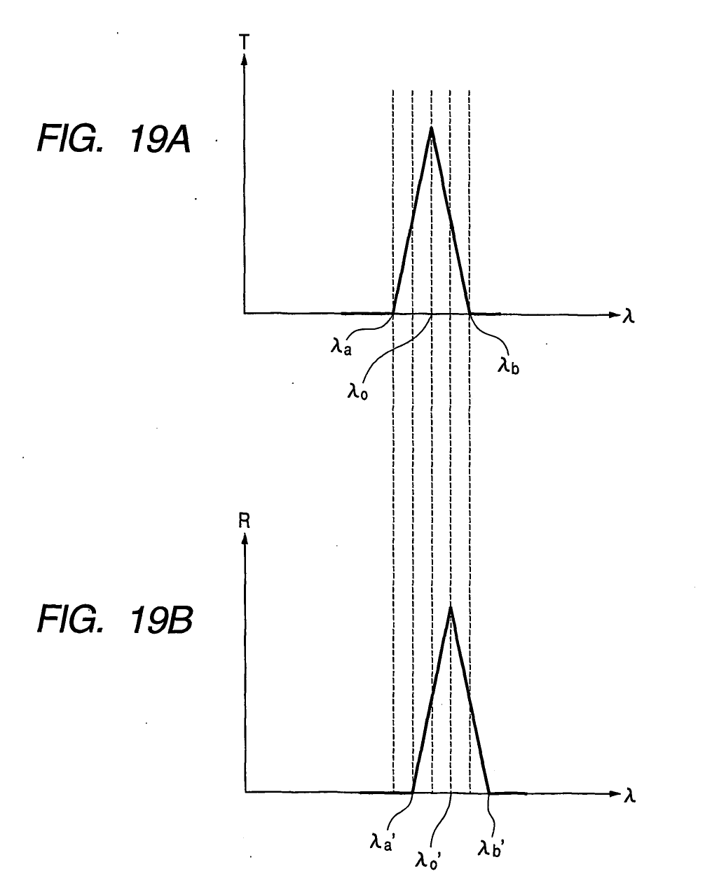

The change in the transmission spectrum by trapping of the target substance by the trapping substance is explained by reference to FIGS. 19A and 19B. FIGS. 19A and 19B show spectrum around the resonance caused by the defect. FIG. 19A shows the spectrum without the trapping, and FIG. 19B shows the spectrum with the target substance trapped. The resonance peak shifts by the trapping. In FIG. 19A, λ₀ is the center frequency of the resonance, and the transmittance region extends in the range between λₐ and λ_{b}. The trapping of the target substance causes shift of λₐ, λ₀, and λ_{b} as shown in FIG. 19A respectively to λₐ′, λ₀′, and λ_{b}′ as shown in FIG. 19B.

Therefore, the presence of the target substance can be detected by detecting the difference shown in FIGS. 19A and 19B. Since the transmittance peak caused by the resonance is sharp, a slight shift of the peak position can be detected, enabling the detection of the trapping with high sensitivity.

For the detection, transmitted light 403 having passed through photonic crystal 1800, or reflected light 405 by the photonic crystal is detected. Light source 402 emits light around the resonance wavelength λ₀. In one method, the spectrum of the transmitted light or of the reflected light is measured to detect the change in the resonance wavelength.

In another method, one wavelength in the range between λₐ and λ_{b} is selected, and the change in the light intensity at the selected wavelength is detected. For example, when a transmitted light is observed at a wavelength between λₐ and λ₀, the shift of the peak wavelength λ₀ to high wavelength side caused decrease of the intensity of the transmitted light at that wavelength.

(Detection Method 3)

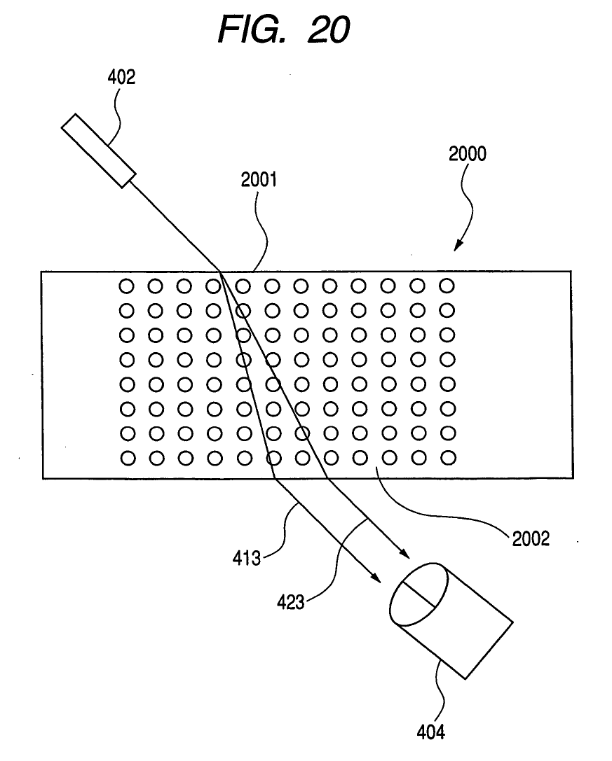

FIG. 20 shows a third method. Photonic crystal structure 2000 contains a trapping substance as shown in FIG. 3, and the plane of the printed sheet face corresponds to the XY plane. Light is introduced from light source 402 through incidence plane 2001. The light is preferably monochromatic and is collimated for high sensitivity, preferably a laser beam. Further, an optical system for collimating the light is preferably provided. A test sample liquid to be inspected is introduced to the vacant structure in the photonic crystal structure. A target substance, an antigen or antibody, present in the test sample liquid is trapped by the trapping substance, changing the photonic band structure. This change causes change in the path of the light traveling through the photonic crystal structure. The traveling direction of the light leaving photonic crystal 401 through emission plane 2002 is not changed regardless of the presence or absence of the target substance, but the position of the emission is changed by the direction of the light traveling in the photonic crystal. FIG. 20 shows light path 413 in the absence of the target substance, and light path 423 in the presence of the target substance. On the extension direction of the optical path, there is placed detector 404 for detecting the change in the optical path. The detector is exemplified by two-division sensor. The quantity of the target substance can be calculated from the outputs of the detector according to a predetermined calculation process.

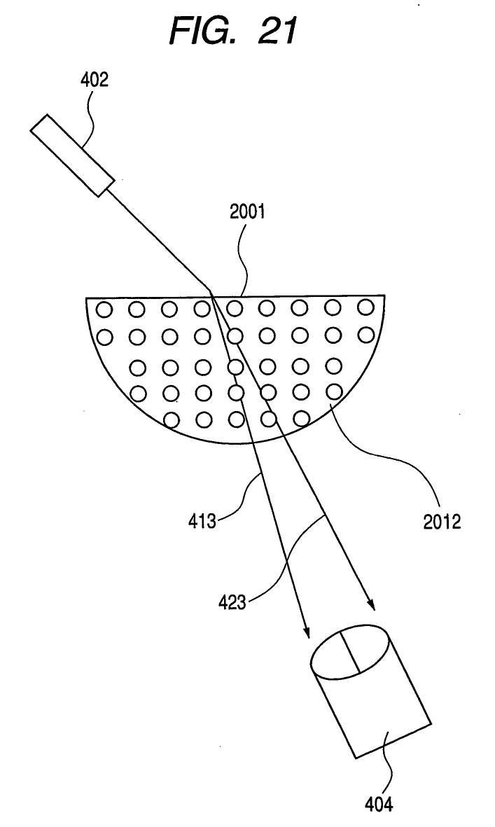

In FIG. 20, the light incidence face and the light emission face are made parallel to each other. Otherwise, as shown in FIG. 21, the light emission face may be made circular with the center at the light incident position (face 2012). With this shape of the light emission face, light leaving the photonic crystal will not be refracted at the light emission face 2012 to keep the light travel direction in the photonic crystal unchanged outside the photonic crystal. Thus the detection sensitivity can be raised by keeping the detector at a longer distance.

On introducing a light beam into a photonic crystal, a slight difference of the incident angle will cause a great change in the light travel direction in the photonic crystal structure. This phenomenon is known as a super-prism effect (Journal of Physical Society of Japan, vol. 55 (2000), March, pp. 172-179). In the photonic crystal structure, at the same incident angle, a slight difference of the wavelength of an incident light beam will cause a great change in the light travel direction.

The super-prism effect occurs in the photonic crystal within a specified region of the incident angle and wavelength of the light beam introduced (Journal of Physical Society of Japan, vol. 55 (2000), March, pp. 172-179).

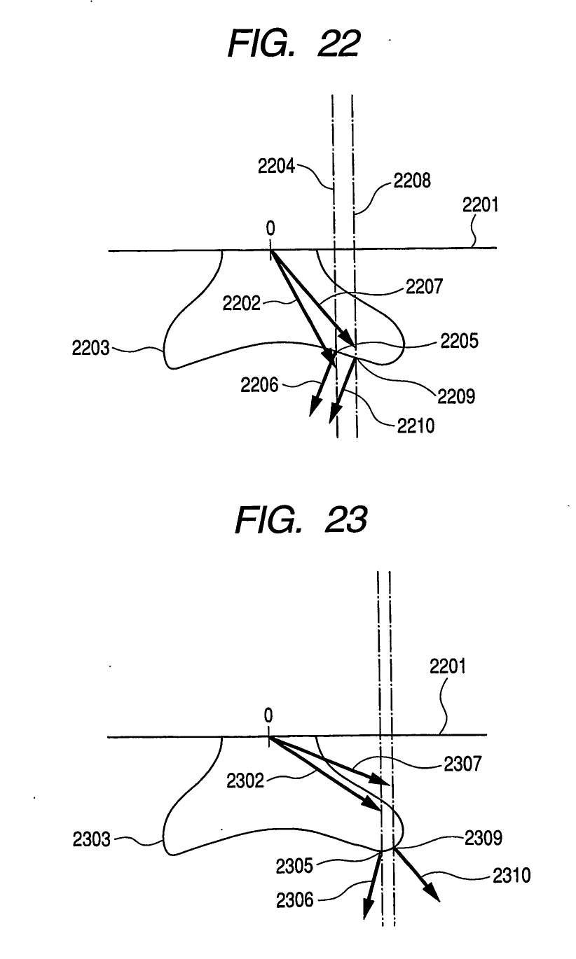

This effect is explained briefly by reference to FIG. 22.

FIG. 22 shows a wave number space. In FIG. 22, the numeral 2201 denotes direction of the interface between the photonic crystal and the outside. The numeral 2202 denotes the wave vector of incident light. The numeral 2003 denotes a constant energy surface having the same energy as the incident energy in the photonic crystal. The numeral 2204 denotes a component parallel to the incident surface of wave vector 2202 of the incident light.

In the photonic crystal, the light energy propagation direction is shown by inclination direction of the energy dispersion face at cross point 2205 of surface 2203 and component 2204. In FIG. 22, the numeral 2206 denotes the inclination direction of the energy dispersion face at cross point 2205, namely the propagation direction of the light energy.

In this case, a slight change in wave vector 2202 does not cause significant change in light travel direction 2206 in the photonic crystal. In FIG. 22, the numeral 2207 denotes a wave vector in the case where the incident angle is changed slightly. In this case, the numeral 2208 denotes a component pf the wave vector 2207 of incident light introduced to the photonic crystal parallel to the interface direction 2201, and intersection 2209 corresponds to intersection 2205. At this point, the inclination of the energy dispersion face shows the light travel direction. Therefore, the light energy propagates in the direction 2210, which is not significantly changed from direction 2206. That is, the light energy propagation direction in the photonic crystal structure is not greatly changed even if the wavelength and direction of the incident light is not changed.

On the other hand, in FIG. 23, incident light of wave vector 2302 is introduced to the same photonic crystal as above (the incident light having the same energy but introduced at a different introduction direction from that of FIG. 22). Intersection 2305 in FIG. 23 corresponds to intersection 2205 in FIG. 22, and the light travels in direction 2306. In the case of FIG. 23, a slight change in the wave vector 2302 to wave vector 2307 moves the intersection to position 2309 and changes the light travel direction to position 2310. As described above, in the photonic crystal, a slight change in the wave vector of the incident light changes greatly the light travel direction.

The significant change in the light energy propagation direction in the photonic crystal structure can be caused also when the photonic band structure is changed slightly. More specifically, a slight change in the photonic crystal structure changes the energy dispersion plane, resulting in change in the position of intersection 2305 and a great change in energy propagation direction. This effect is called a super-prism effect. For obtaining this effect, the constant energy surface at intersection 2205 or 2305 has necessarily a large curvature.

As described above, by selecting the wave vector of the incident light, a slight change in the photonic crystal structure changes greatly the light travel direction. Therefore, at such an incident direction, the target substance trapped in the photonic structure can be detected with a high sensitivity.

5. Arrangement of Photonic Crystal

The arrangement of the photonic crystal in the fluid flow path is explained below.

A. Arrangement for Projecting Light to Cross Flow Path

A-1. Two-dimensional Plane Directed Parallel to Flow Path

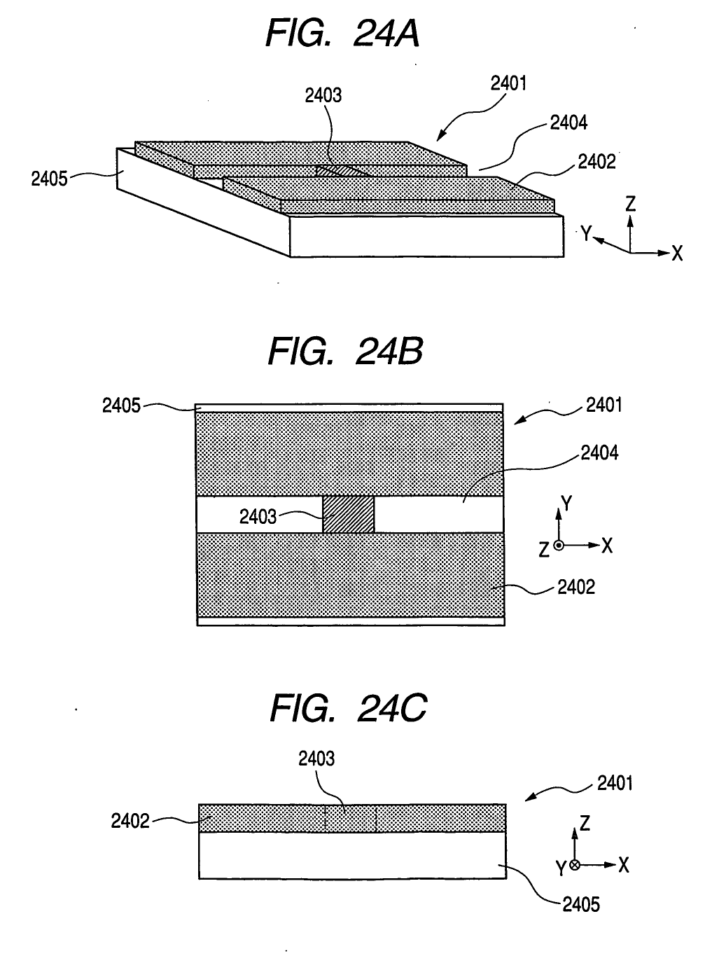

FIGS. 24A-24C illustrates schematically a sensor chip 2401 placed in a portion of a flow path for passing test sample liquid through a periodic structure, namely a photonic structure of the present invention. The sensor chip is constituted of connecting flow path 2404 and two-dimensional photonic crystal 2403 having a continuous vacant structure and columnar structure therein as shown in FIG. 3.

Sensor chip 2401 is produced by working by a semiconductor processing technique a silicon layer of 500 nm thick on insulating layer of 1 μm thick on an SOI (silicon on insulator) substrate.

Region 2404 between two side walls 2402 serves as the flow path. Flow path side walls 2402 are formed by photolithography. In FIGS. 24A, 24B, and 24C, the fluid is passed through flow path 2404 in the X direction (hereinafter referred to as “flow direction” or “flow path direction”). In FIG. 24A, the top side of flow path 24004 opposite to the bottom constituted of insulation layer 2405 is shown to be open, for convenience of explanation. Actually, however, the flow path is covered with a glass or resin plate not to leak the fluid. In other drawings also, the cover is not shown.

The surface of the sensor chip parallel to the substrate is called a “sensor chip face”. The distance between the cover plate and the substrate is referred to as a “height of flow path”, and the distance between two side walls is called “width of the flow path”.

The both ends of flow path 2404 are connected to other flow paths or other structures for extraction of the fluid or separation of the components, or the like.

Photonic crystal 2403 in flow path 2404 has a photonic band gap for TE polarized light. It may be constituted of columnar solid portions and an empty portion as the photonic crystal 100 in FIG. 3, or may be constituted of a continuous body and holes arranged periodically as shown in FIGS. 9A and 9B. The sensor chip is prepared by EB (electron beam) drawing, development, and dry-etching. A trapping substance such as an antibody is supported on the surface of the solid portion.

A test sample fluid composed of water and an antigen dissolved therein is passed through flow path 2404. During the passage of this fluid through photonic crystal 2403, the antigen bonds specifically to the supported antibody by antigen-antibody reaction and is immobilized. This changes the photonic band structure of the photonic crystal, causing change in light transmission property to the TE polarized light wavelength. For example, in FIG. 2, the band edge 2001 shifts to the short wavelength side. By comparing the transmitted light intensity after the antigen-antibody reaction with that before the reaction, the target substance, the antigen in this example, can be detected.

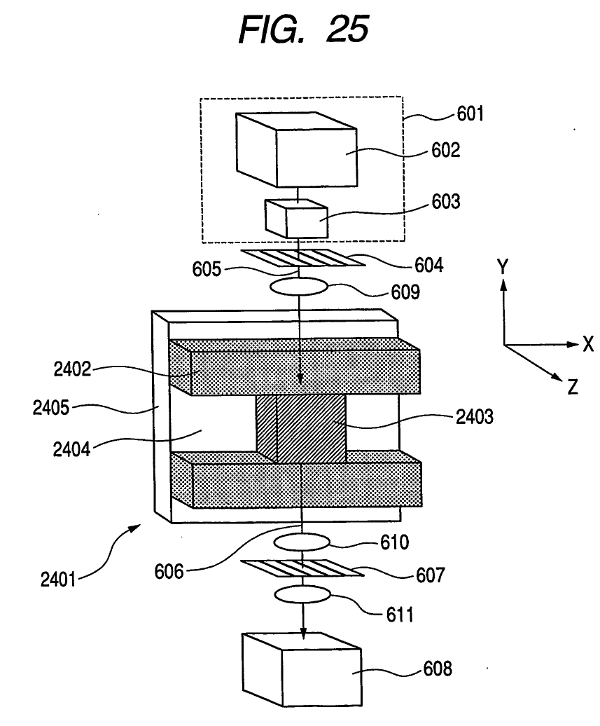

FIG. 25 shows an example of the entire constitution of the device of the present invention in which an optical system is placed to project the measuring light onto the sensor chip and to detect the change in the properties. Two-dimensional photonic crystal 2503 of the sensor chip has a periodic structure, and is constituted of solid structure members as shown in FIG. 3, the long axis direction being perpendicular to the bottom face of the flow path. The sensor chip face is parallel to the XY plane which is parallel to the two-dimensional plane. The direction of the flow of the test sample liquid is defined as the X axis direction. Electromagnetic wave-projecting means 601 comprising laser 602 for generating electromagnetic wave and optical system 603 emits laser beam 605 of a wavelength of 1550 nm. The TE mode thereof is selected by polarizing plate 604. The TE-polarized light having a wavelength of the photonic band edge is focused by condenser lens 609 onto the side face of photonic crystal 2503 and is projected onto flow path side wall 2402 of sensor chip 2401 to enter photonic crystal 2503. Therefore, the light is introduced in the Y direction. That is, in this embodiment, the two-dimensional plane (XY plane) of the photonic crystal is parallel to the light incident direction (Y axis) and parallel to the liquid flow direction (X axis). The light having been transmitted through the photonic crystal is emitted from the side wall of a second flow path side wall 2402. This light (signal light) 606 is collimated by lens 610, and only the TE component of the polarized light is taken out through polarizing plate 607, a polarization controlling means. This polarized light component is condensed by condenser lens 611 and is detected by photodiode 608, an electromagnetic wave detector.

The transmittance of the light at 1550 nm, which corresponds to the band edge of photonic crystal 2503, is changed by the antigen-antibody reaction. Therefore, the antigen, a target substance, can be detected by measuring the light transmittance before and after the reaction.

After immobilization of the antigen by the antibody, the light transmittance may be measured after washing of the flow path and the photonic crystal with water or the like, or may be measured without the washing.

The change in the intensity of the transmitted light at a certain time after start of the flow of the liquid depends on the concentration of the antigen in the fluid. Therefore, the concentration of the antigen in the fluid can be determined by measuring the change in the transmitted light intensity by normalization by the time.

In the constitution of this embodiment, the periodic structure of the two-dimensional crystal is constituted of solid constructing members as shown in FIG. 3 and the two-dimensional surface of the periodic structure is laid parallel to the flow, so that the interspace is sufficiently large, and the test sample liquid is allowed to flow without stagnation. This enables the design of the flow sectional area to be smaller, making the sensor chip compact. In addition, the light incidents parallel to the two dimensional surface and perpendicular to the flow, making size of the total sensor apparatus further compact.

In this embodiment, a specific reaction between the target substance and the trapping substance is utilized, whereby an intended specific target substance only can be selectively detected even with a sample fluid containing plural substances.

A-2. Two-dimensional plane Directed Perpendicularly to Flow Path

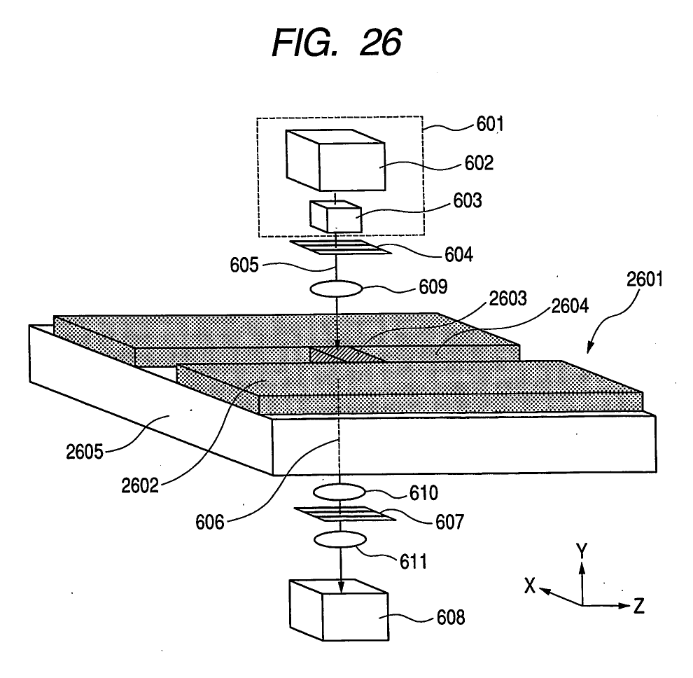

Another embodiment of the sensor of the present invention is explained by reference to FIG. 26.

Sensor chip 2601 is formed from an acrylic resin by molding, in which photonic crystal 2603 is placed in a portion of flow path 2604 constituted by flow path side walls 2602 on lower layer (insulation layer) 2605.

Photonic crystal 2603 has a structure in which holes as the vacant structure are arranged periodically in a solid as shown in FIGS. 9A and 9B. In photonic crystal 2603, holes are arranged to have the long axis of the hole directed parallel to flow path 2604 to allow the test sample liquid to flow through the holes. In FIG. 26, the plane parallel to the two-dimensional plane is taken as the XY plane, so that the flow of the test sample liquid is perpendicular to the XY plane and parallel to the Z axis.

Optical system 601 to 611 for the measurement is placed separately above and below sensor chip 2601. The light is projected to photonic crystal 2603 in a direction perpendicular to substrate 2605 and the sensor chip face. The sensor chip face is parallel to the ZX plane, so that the light is projected in the Y axis direction. Therefore, the two-dimensional direction (XY plane) of the photonic crystal of this embodiment is parallel to the light projection direction (Y axis) and perpendicular to the flow direction (Z axis). Polarizing plate 604 is placed to introduce allow the light of the TM mode into photonic crystal 201.

Photonic crystal 2603 is designed to have photonic band gap to TE-polarized light between the first band and the second band in the photonic band structure, with the wavelength of the first band edge corresponding to about 1550 nm. The antigen-antibody reaction causes change in the photonic band structure to change in the transmission property to the wavelength of TE-polarized light. For example, in FIG. 2, the band edge 2001 is shifted to the short wavelength side. By comparing the light transmission intensity after the antigen-antibody reaction with that before the reaction, the target substance, the antigen in this example, can be detected.

By passing the test sample liquid through the photonic crystal, the antigen-antibody reaction occurs. For detection of the antigen, light beam 605 of wavelength of 1550 nm is projected to the photonic crystal from electromagnetic wave-projecting means 601 comprising laser 602 and optical system 603 through polarizing plate 604 as the polarization controlling means by condensing the light by lens 609 to focus the light on the surface of photonic crystal. Light 606 having passed through photonic crystal 2603 is collimated by lens 610 and is allowed to pass polarizing plate 607. The light is collected by lens 611 and is detected by photodiode 608. In the similar manner as in Embodiment A-1, the antigen can be detected by comparing the transmitted light intensity after the antigen-antibody reaction with that before the reaction.

In this embodiment, the two-dimensional photonic crystal has a hole structure as shown in FIGS. 9A and 9B. To pass the test sample liquid without stagnation, the length of the hole, namely the thickness of the periodic structure of the photonic crystal, should be made small in the long axis direction depending on the viscosity of the test sample liquid. On the other hand, since the two-dimensional plane (XY plane) of the periodic structure is perpendicular to the flow path, namely to the flow of the test sample liquid, the light for the detection can be projected in the direction parallel to the two-dimensional plane and perpendicular to the sensor chip plane. As the result, the distance between the light source to the detector can be made shorter, whereby the entire device can be made more compact than that of embodiment A-1.

A-3. Detection by Measurement of Reflected Light

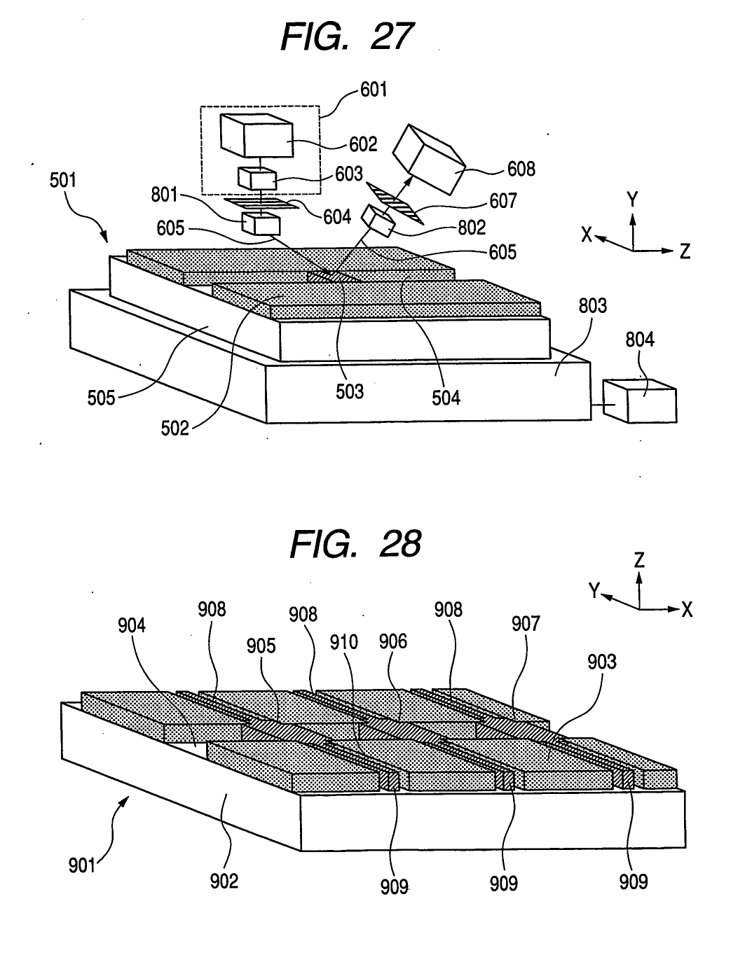

FIG. 27 shows an embodiment of the present invention in which the change in the intensity of the reflected light from the photonic crystal is measured. The optical detection system comprising photodiode 608 is placed at the same side of the light source system above the substrate. Additionally, there are aligning means 801,802, temperature controller 804, temperature-controlling means 803 connected to the temperature controller. Otherwise the constitution is the same as in Embodiment A-2.

Both the incident light and the reflected light are on the XY plane, and the light is projected and reflected at a prescribed angle. The wavelength of laser light source 603 and the polarization direction of the polarizing plate 604 are adjusted so that the light corresponding to the band edge of photonic crystal 2603 may be projected in the TE mode.

Photonic crystal 503 is a porous structure as shown in FIGS. 9A and 9B, wherein the longitudinal axes of holes are located parallel to the flow path direction, i.e. in the Z-axis direction.

On the other hand, light 605 in TE mode is a polarized light having an electric field component in Z-axis direction in the figure.

Photonic crystal 503 may consist of a structure comprised of columns as shown in FIG. 8. In this case, photonic crystal 503 is located so as to direct the columns parallel to Y-direction in FIG. 27 and the polarizing plate 604 is set to make incident light 605 have the electric field component in XY plane.

In the constitution of A-1, A-2, or A-3, the photonic crystal may be replaced with one of the one-dimensional structure as shown in FIG. 6. For smooth flow of the test sample liquid without stagnation, the flow path is provided to be parallel to thin films 102 of the thin film structure, and the light is projected to thin film 102 perpendicularly or at a prescribed angle.

Otherwise, the photonic crystal may be replaced with the one of three-dimensional structure as shown in FIG. 7. The three-dimensional photonic crystal constituted by stacking spherical constructing members 301 can be placed in any direction to the flow path since the test sample liquid flows through interstice 302 thereof.

B. Projection of Light Parallel to Flow Path

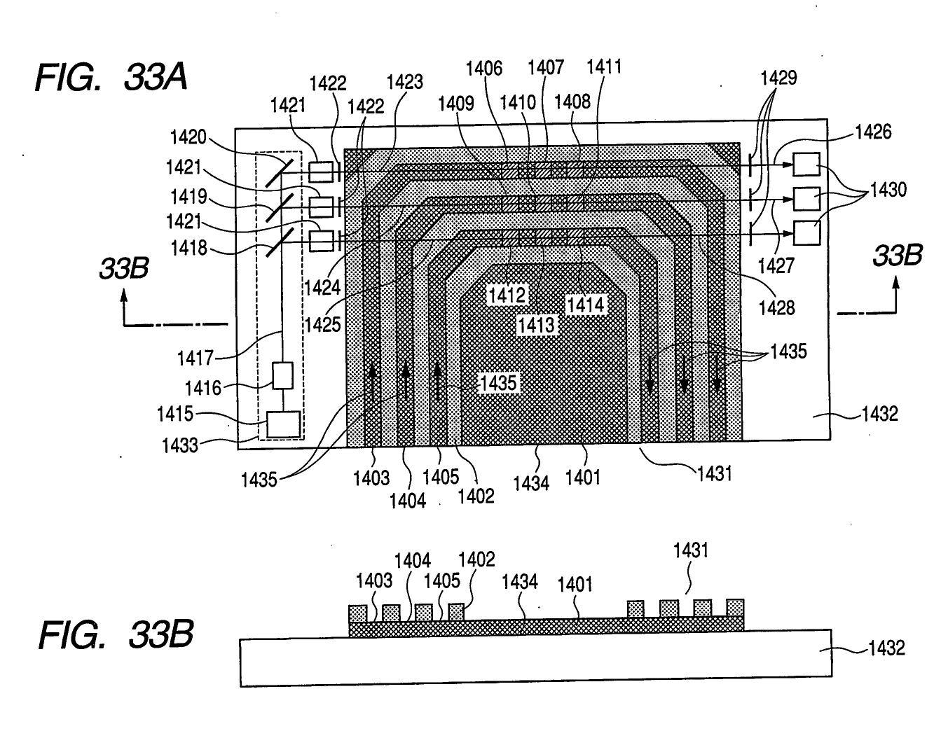

The light may be introduced into the flow path, passed through the flow path, and introduced into a photonic crystal. This will be explained later specifically in the description of the embodiment. Briefly, as shown in FIGS. 33A and 33B, the flow path is bent at an angle of 90° at an upstream side and a downstream side of the photonic crystal; an external light is introduced at the one bend portion; and after penetration of the photonic crystal, the light is taken out from the other bend portion.

In this constitution, the optical path is provided along the length direction of the liquid flow path. This makes difficult shortening of the optical path. However, plural sensors may be placed in the flow path from the upstream side to the down stream side, and measurement can be made with the plural sensor with a single light source, advantageously.

6. Constitution with Plural Sensors

With a test sample liquid containing plural target substances, the respective target substances can be detected simultaneously by placing plural photonic crystals carrying respectively a trapping substance specifically reactive to the target substances in the liquid flow path and conducting the measurements.

Various constitutions of the sensors are possible by arrangement of the plural photonic crystals, as explained below.

C. Serial Arrangement of Photonic Crystals in Flow Path

C-1. Measurement with Plural Light Sources

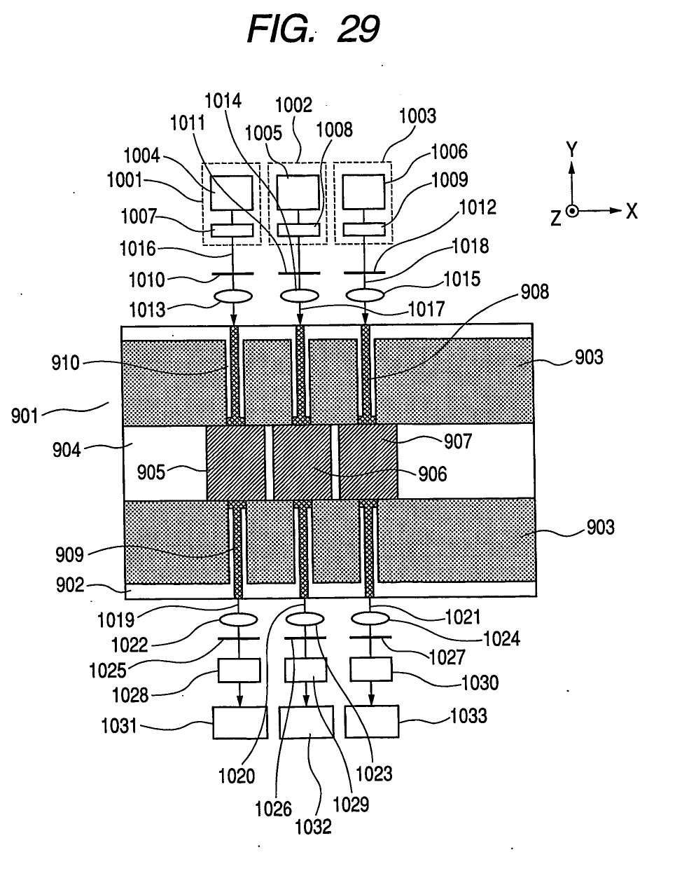

FIGS. 28 and 29 illustrate a sensor having plural photonic crystals of FIG. 25 explained in Section A-1 arranged in series in a flow path. Herein, the term “series” signifies the state of arrangement along the flow path from an upstream side to a downstream side. The arrangement need not be strictly in a straight line in the flow path directions, and may be selected to meet the structure, and the arrangement of the optical measurement system.

FIG. 28 shows sensor chip 901 having three photonic crystals in series in a flow path.

This chip can be prepared by forming barrier walls 903 to provide flow path 904 therebetween, optical waveguides 908,909 of 5 μm in width, and regions for positioning photonic crystals 907 by photolithography on an insulation layer 902 of 2 μm thick of an SOI substrate, and forming photonic crystal structure by EB lithography.

Photonic crystals 905,906,907 have a structure having silicon columns arranged two-dimensionally in a triangle lattice like the ones shown in FIG. 8. The photonic crystals 905, 906, 907 are in a size of 100 μm in length, 100 μm in width, and 1 μm in height. Flow path 904 are in a size of 100 μm in width and 1 μm in height.

Photonic crystals 905,906,907 carry respectively different kind of antibody, and are designed to have a band gap at different wavelength region in that state and to employ different wavelength of the band edge for the detection.

The three photonic crystals carry respectively an antibody different in the kind on the surface of each of the vacant structures. The respective photonic crystal detect different kinds of antigens. Therefore this one chip is capable of detecting three kinds of antigens. For confining the light in the optical waveguide, optical waveguides 908,909 and barrier wall 903 are separated by a spacing of 2 μm.

FIG. 29 shows constitution for detecting plural target substances by employing the aforementioned sensor chip. Three optical detection systems are provided for the three photonic crystals.

A first optical detection system is constituted of electromagnetic wave-projecting means 1001 comprising semiconductor laser 1004 and optical system 1007, polarizing plate 1010 as a polarization controlling means, lens 1013, lens 1022, polarizing plate 1025 as a polarization controlling means, aligning means 1028, and photodiode 1031.

A second optical detection system is constituted of electromagnetic wave-projecting means 1002 comprising semiconductor laser 1005 and optical system 1008, polarizing plate 1011 as a polarization controlling means, lens 1014, lens 1023, polarizing plate 1026 as a polarization controlling means, aligning means 1029, and photodiode 1032.

A third optical detection system is constituted of electromagnetic wave-projecting means 1003 comprising semiconductor laser 1006 and optical system 1009, polarizing plate 1012 as a polarization controlling means, lens 1015, lens 1024, polarizing plate 1027 as a polarization controlling means, aligning means 1030, and photodiode 1033.

The first, second, and third detection systems correspond respectively to photonic crystals 905,906,907. Polarizing plates 1010,1011,1012 polarizes the light to TE polarized light to the sensor tip face.

Semiconductor lasers 1004,1005,1006 generate respectively light of the wavelength around the center of the band edge region of the photonic band gap in the photonic band structure of photonic crystals 905,906,907 carrying respectively different kind of antigen. For this purpose, semiconductor lasers 1004,1005,1006 generate respectively a different wavelength of light.

Three laser beams 1016,1017,1018 generated by magnetic wave-generating means 1001,1002,1003 travel through the lenses and three optical waveguides 908 to photonic crystals 905,906,907. The three light beams leaving the three photonic crystals travel through the other three optical waveguides 909 and are allowed to emit as signal light beams 1019,1020,1021 out of the sensor chip. The light beams are respectively measured by photdiodes 1031,1032,1033.

This constitution requires a light source and an optical system for the respective photonic crystal. However, in this constitution the photonic crystals 905-907 can be replaced by one common photonic crystal advantageously. Further, since the respective optical waveguides are independent, the band gaps can be independently selected, and the trapping substances for the detection can be selected independently advantageously. Naturally, the photonic crystals need not have the structure shown in FIG. 8, but may be replaced partly or entirely with the constitution described in Section A-2 or A-3.

The property of the photonic crystal is changed by flow of the fluid containing the antigen by an antigen-antibody reaction. Thereby, the light transmission index is changed at the band edge. Therefore, three kinds of antigens can be detected simultaneously by measuring the changes in the transmitted light intensities by the antigen-antibody reactions in the respective photonic crystals 905,906,907 at the respective band edge regions. The kind of the antigen to be detected can be changed by selecting the antibody.

In this embodiment, even if the fluid contains an additional substance other than the three antigens specifically bonding to the three antibodies carried by the three photonic crystals, the objective three antigens can be selectively detected, since the additional substance does not bond specifically to the antibody carried by the photonic crystal.

C-2. Measurement with Single Light Source

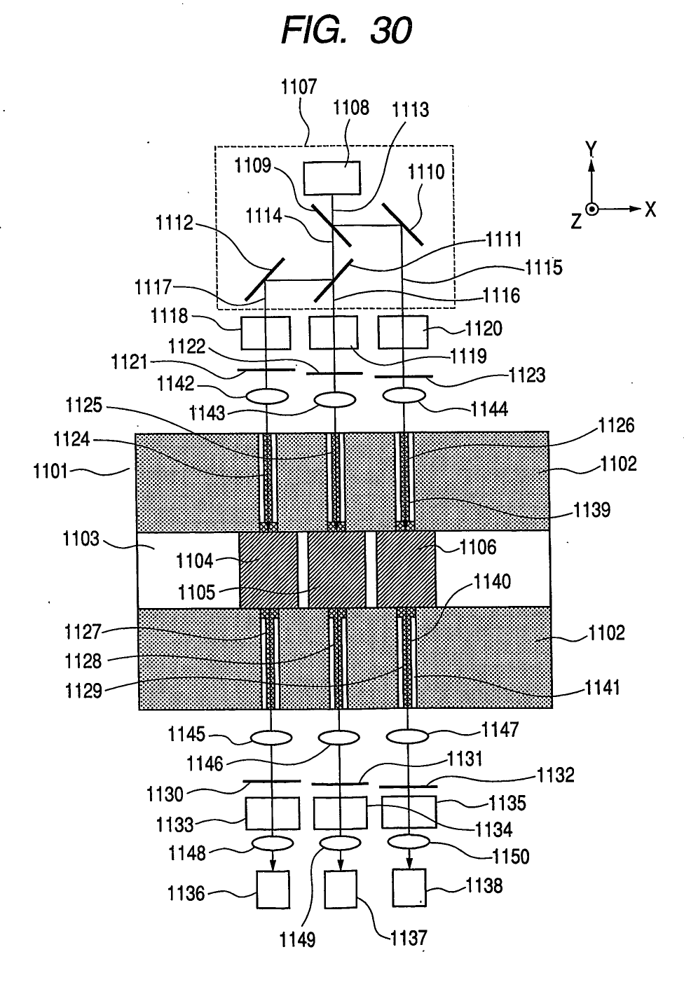

FIG. 30 shows an example of the sensor of the present invention in which plural target substances are detected with one electromagnetic wave-generating means.

Sensor chip 1101 is constituted of barriers 1102; optical waveguides 1139,1140; gaps 1141 between the optical waveguides and the barriers; flow path 1103; and photonic crystals 1104,1105,1106.

Photonic crystals 1104,1105,1106 have the same structure as those shown in FIG. 8. However, the photonic crystals 1104,1105,1106 are designed respectively to have the structures in which the photonic band gap edges are at nearly the same wavelength in a state that different kinds of antibodies are carried on the surfaces of the vacant structure members. For obtaining the same band edges with different antibodies carried, the photonic crystals should have different periodic structures.

Electromagnetic wave-emitting means 1107 is constituted of laser 1108; beam splitter 1109 for splitting one laser beam from the one laser into three laser beams; mirror 1110; beam splitter 1111; and mirror 1112. The wavelength of the light beam emitted by laser 1108 is adjusted to coincide with the band edge of the photonic crystals for the detection.

In this embodiment, the semiconductor laser apparatus has an annexed resonator. One laser beam 1113 emitted from this semiconductor laser is split into three laser beams 1115,1116,1117. The three laser beams are emitted from the electromagnetic wave-projecting means, and are introduced respectively through aligning means 1118,1119,1120, polarizing plates 1121,1122,1123 as the polarization-controlling means, and lenses 1142,1143,1144 to three optical waveguides 1139 on one side of sensor chip 1101.

The light beams introduced to three optical waveguides 1139 penetrate three photonic crystal, travel through optical waveguides 1140 on the other side, lenses 1145,1146,1147, polarizing plates 1130,1131,1132 as the polarization-controlling means, lenses 1148,1149,1150 to reach spectrometers 1133,1134,1135, and are subjected to measurement with spectrometers 1136,1137,1138.

For detection of an antigen, a fluid containing the antigen is passed through the photonic crystal, whereby the antigen is trapped by bonding to the antibody carried on the surface of the vacant structure of the photonic crystal by an antigen-antibody reaction. This causes shift of the band edge of the photonic band gap in the photonic band structure of the photonic crystal to change the light transmittance. The antigen is detected by this change.

With the sensor constitution of this embodiment, three kinds of antigens as the target substances can be detected concurrently. From the change in the light transmittance, the kind of the antigen can be identified, or the concentration thereof can be estimated.

In this embodiment, the photonic crystals should have the same band gap edges. Thereby, the common light source can be employed, and the same type of optical system including the detection system can be employed.

FIGS. 33A and 33B show another example of serial arrangement. FIG. 33B is a sectional view taken along line 33B-33B in FIG. 33A.

In FIGS. 33A and 33B, sensor chip 1432 has three flow paths which have respectively three photonic crystals carrying different antigens are placed in series. Thereby, nine kinds of antigens specifically capable of bonding to the nine antibodies can be detected in the test sample liquid flowing through the flow path.

The three flow paths 1403,1404,1405 are formed on insulation layer 1401 on substrate 1432 by partitioning by side walls 1402. The flow paths are bent twice at an angle of 45°, totally 90° before the photonic crystal positions, and bent also twice at an angle of 45°, totally 90° after the photonic crystal positions.

A laser beam 1417 emitted from wavelength-variable laser 1415 is passed through beam shaper 1416 for decreasing the beam diameter, split into three beams by beam splitters 1418,1419 and mirror 1420, passed through aligning means 1421 and polarizing plates 1422, and introduced through 90° bend portions into three flow paths 1403,1404,1405. The respective light beams 1423,1424,1425 travel parallel to the flow paths, passed through serially arranged photonic crystal sets (1406,1407,1408), (1409,1410,1411), and (1412,1413,1414), and emitted outside through other 90° bend portions. The emitted light beams are respectively introduced as signal light beams 1426,1427,1428 through polarizing plate 1429 into three detecting means 1430.

In this embodiment, sensor chip 1434 and optical detection system are mounted on one and the same substrate 1432. The both ends of the flow paths of sensor chip 1434 are connected to flow paths outside the sensor. As shown in FIGS. 33A and 33B, sensor chip 1434 has three flow paths, and in each of the flow paths, three photonic crystals are placed, each photonic crystals carrying different kinds of antibodies on the vacant structure surfaces of the solid portion.

The three photonic crystals placed in each of the flow paths are designed not to cause overlap of the wavelength region of the photonic band gaps. The photonic crystal in another flow path may have photonic band gap having wavelength region overlapping with that of the above photonic crystal. Arrows 1435 denote the directions of the flow of the fluid.

The variable wavelength range of the wavelength-variable laser covers all of the band edge wavelength ranges of the nine photonic crystals employed for the detection. Three signal light beams 1426,1427,1428 are passed respectively through polarizing plates 1429, and subjected to measurement with spectrometer 1430.

The measurement can be conducted as follows. Wavelength-variable laser 1415 emits a light beams of scanning wavelength. The light transmittance is measured at the band edge wavelength of each of the photonic crystal in each of the flow paths. The target substance trapped by the photonic crystal shifts the band edge wavelength. This is detected by change in the light transmittance. Since, in the one and the same flow path, the respective band edges are outside the band gaps of the other photonic crystals, the changes of the respective band edge wavelengths can be measured without interruption of the light travel by another photonic crystal in the same flow path.

In the constitution of this embodiment, the band edge wavelengths of the photonic crystals need not be the same as that of another flow path since the measurement is conducted by wavelength scanning by wavelength-variable laser. Therefore, the photonic crystal need not be designed for each of the flow paths, but is designed so as not to cause overlap of the band gap in the one flow path. Therefore, common photonic crystals may be used even if the flow paths are increased. Further, since the signal lights of the different flow paths are detected by separate detectors 1430, the signal light can be differentiated even when the band edge wavelengths are the same between the flow paths.

By scanning of the wavelengths of the laser beam, the change is measured of the spectrum of the signal light beam caused by the antigen-antibody reaction by means of spectrometer 1430. Thereby, the nine antigens can be detected concurrently. More target substances can be detected by increasing the number or the flow paths and the number of the detection systems.

D. Arrangement of Photonic Crystal in Parallel in Flow Path

The sensor of the present invention may have plural photonic crystals placed in parallel in one a flow path. Herein, the term “parallel” signifies arrangement of plural photonic crystals in the direction of the breadth of the flow path. The arrangement direction need not be strictly perpendicular to the flow direction, insofar as the projected measuring light beam crosses the flow path and travels successively through all of the photonic crystals. This will be understood from the explanation below.

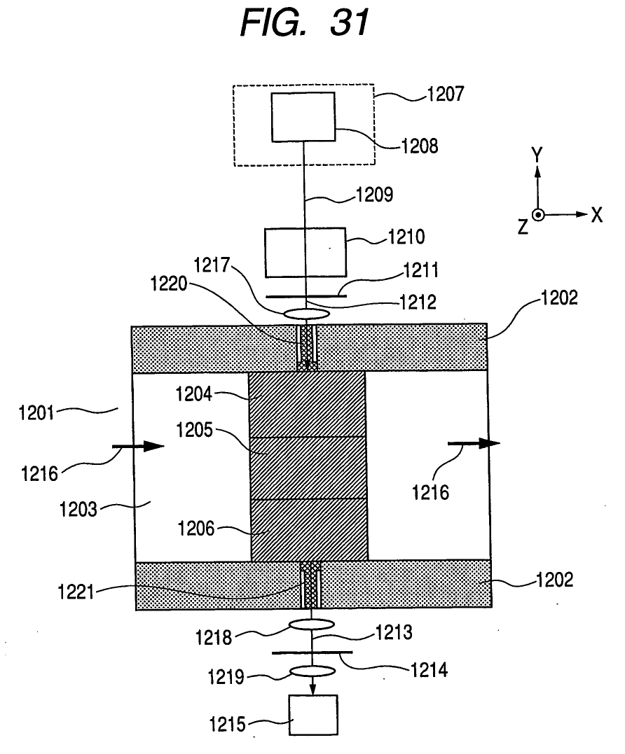

FIG. 31 shows an example of parallel arrangement of three photonic crystals.

Sensor chip 1201 has a flow path of 100 μm in width and 1 μm in height between side walls 1202. Three photonic crystals having a columnar structure of FIG. 8 are placed in parallel at a portion of the flow path with the column-supporting substrate faces being directed in the breadth direction of the flow path. The optical path in the photonic crystal lies in the XY plane and penetrates all the photonic crystals. Arrows 1216 show the direction of the flow of the fluid.

Any type of the photonic crystals employed in the examples above may be used in this embodiment. With the photonic crystals having the structure of FIG. 8, the photonic crystals are arranged such that the two-dimensional plane (XY-plane) is directed parallel to the light introduction direction (Y axis) and parallel to the flow direction (X axis) as shown in FIGS. 24 and 25, and the column-supporting substrate faces are on one plane. With the photonic crystals having the hole structure of FIGS. 9A and 9B, the photonic crystals are arranged such that the two-dimensional plane (YZ-plane) is directed parallel to the light introduction direction (Y axis) and perpendicular to the flow direction (X axis).

Three photonic crystals 1204,1205,1206 are designed so that the photonic band gap regions will not overlap when three photonic crystals respectively carry different antibody. For instance, the band gap of photonic crystal 1204 ranges from 1350 nm to 1400 nm; the band gap of photonic crystal 1205 ranges from 1450 nm to 1500 nm; and the band gap of photonic crystal 1206 ranges from 1550 nm to 1600 nm. Without the overlap of the band gaps, the light beam having a wavelength of the band gap of one photonic crystal transmits through other optical crystals. Therefore, the shift of the respective band gap edges can be measured independently of the presence of other photonic crystal.

Wavelength-variable laser 1108 is employed as the light source. The wavelength range of wavelength variable laser 1108 covers all of the band gap edge wavelengths.

Laser beam 1209 emitted from light (electromagnetic wave)-emitting means 1207 including laser 1208 is introduced through aligning means 1210, polarizing plate 1211, and lens 1212 into optical waveguide 1220. The light transmitted through optical waveguide 1220 is introduced into photonic crystal 1204. The light having been transmitted through photonic crystal 1204 is introduced to photonic crystal 1205. The light having transmitted through photonic crystal 1205 in introduced into photonic crystal 1206. The light having transmitted all the photonic crystals is introduced into optical waveguide 1221 and emitted outside as signal light beam 1231.

This signal light beam 1231 is transmitted through lens 1218, polarizing plate 1214, and lens 1219, and is measured with spectrometer 1215 as the spectrum measuring means. The spectrometer includes a CCD detector equipped with a spectroscope and an optical spectrum analyzer.

The measurement is conducted by wavelength-scanning with the wavelength-variable laser, or by switching the detection wavelength between the band gap edge wavelengths of the three photonic crystals.

Firstly, in the measurement, when the target substance has been trapped, the band gap edge is shifted, which changes the transmittance of the light at the band gap edge wavelength of photonic crystal 1204. The light which is transmissive before the trapping is intercepted, if the wavelength of the light is brought into the band gap range by the shift caused by the trapping. Since this change occurs out of the band gaps of other photonic crystals 1205,1206, this change is considered to be due to the structure change in the band of photonic crystal 1204.

Secondly, the wavelength of the light beam from the light source is switched to the band edge wavelength of photonic crystal 1205. Assuming that this wavelength of light is transmissive before the detection, drop of the transmittance of the light signifies the change in the band structure of the photonic crystal 1205.

Further, the measurement is conducted similarly by changing the wavelength to the band gap wavelength of photonic crystal 1206. In such a manner, the change in the properties of the three photonic crystals can be detected.

This constitution should be designed not to cause overlap of the band gaps of the photonic crystals, but requires only one optical path for the optical measurement system and only one detection system.

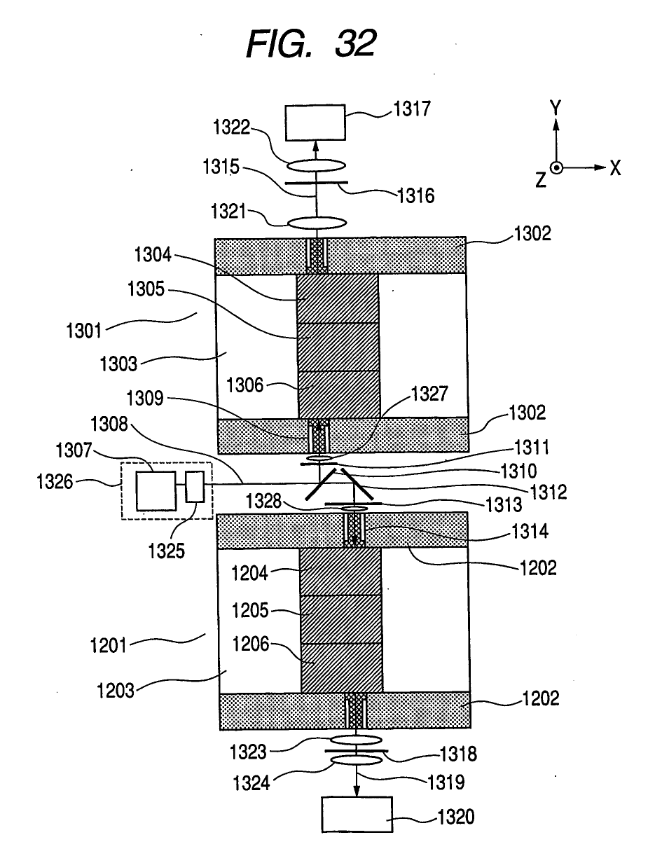

FIG. 32 shows another example of the constitution employing plural sensor chips arranged in parallel for detecting more kinds of target substances.

Sensor chips 1201,1301 have respectively the same constitution as that in FIG. 31, but the two sets of the photonic crystals 1204,1205,1206 and 1304,1305,1306 are placed on separate flow paths. The one light source is used commonly. The light beam is split by half mirror 1310, and introduced into respective photonic crystals placed on the flow paths.

The kinds of antibodies carried by the photonic crystals 1204,1205,1206,1304,1305,1306 are different. The photonic crystals 1204,1205,1206 carrying the antibodies are designed not to cause overlap of the photonic band gaps, and the photonic crystals 1304,1305,1306 carrying the antibodies are designed not to cause overlap of the photonic band gaps. The band gaps of the photonic crystals in separate parallel arrangement sets may overlap.

Light beam 1308 from electromagnetic wave-emitting means 1326 constituted of broad band light emission diode 1307 and optical system 1325 for collimating the light is split into two beams 1309,1314. As the broad band emission diode, an SLD (Super Luminescence Diode) is employed.

Light beam 1309 is introduced through polarizing plate 1311 and lens 1327 into sensor chip 1301, and light beam 1314 is aligned by mirror 1312 and is introduced through polarizing plate 1313 and lens 1328 into sensor chip 1201. Signal light beams 1315,1319 from the sensor chip are measured finally by spectrum detectors 1317,1320 as the spectrum measuring means similarly as in Embodiment C-2.

In such a manner, by using different kinds of antibodies carried by respective photonic crystals, six kinds of antigens can be concurrently detected by measuring the change in the spectrum of signal light beam from each of sensor chips 1201, 1301 caused by the antigen-antibody reaction by means of spectrum detector 1320,1317 corresponding to sensor chips 1201,1301.

E. Combination of Serial Arrangement and Parallel Arrangement

From the above descriptions regarding the serial arrangement and parallel arrangement, naturally, plural sets of parallel arrangement of photonic crystal sets may be arranged in series in one flow path. It may be also possible to arrange one or more photonic crystals in series, following at least one of photonic crystals constituting a parallel arrangement in one flow path.

7. Fiber-Employing Sensor

A holey fiber is known which has empty tubular holes in the portion corresponding to cladding portion of an optical fiber to decrease the effective refraction index of that portion, disclosed in U.S. Pat. No. 6,334,019. This fiber, which may be constituted of a core portion and a cladding portion made from the same material, is noticed as a novel optical fiber in comparison with conventional optical fiber utilizing total reflection of electromagnetic wave at the interface between a core portion of a higher refractive index material and a cladding portion of a lower refractive index material. The solid portion is formed from glass or plastic material. The holey fiber has the continuous holes formed in the length direction, and the cross section of the holey fiber has a solid core and a hole region around the core. In one type of hole region, the holes are arranged regularly, and in another type of hole region the holes are distributed randomly. The holey fiber which has regular periodic arrangement of holes in the radius direction and has a band gap similarly as the photonic crystal is called a photonic crystal fiber.

The photonic crystal fiber has holes arranged at regular intervals in the radius direction at a cross section perpendicular to the fiber length direction. The periodic structure gives a photonic band gap, which confines an electromagnetic wave within the radius direction to transmit it through the fiber without considerable loss.

This holey fiber can be employed as the photonic crystal fiber in the sensor of the present invention.

For using the holey fiber as the sensor, preliminarily, a trapping substance capable of reacting selectively with a target substance to be detected is allowed to adhere onto the internal surface of the tubular holes, and a fluid containing the target substance, namely a test sample liquid is allowed to flow through the holes. When the test sample liquid is allowed to flow through the tubular holes, the target substance in the fluid will react selectively with the trapping substance supported on the inside surface of the holey fiber, changing the property of the holey fiber, specifically light transmittance or reflectivity of the specific wavelength of an electromagnetic wave.

Accordingly, the target substance can be detected or concentration thereof can be quantitatively determined by measuring the change in the signal electromagnetic wave caused by the bonding reaction. The holey fiber comprises all of the necessary parts for the series of the sensing operation, enabling effective and simple sensing.

In particular, by use of the holey fiber as the photonic crystal fiber, change in signal electromagnetic wave caused by the bonding reaction of the target substance to the trapping substance can be detected with high sensitivity.

The holey fiber itself may be used as the entire flow path. Otherwise, the holey fiber may be placed in a part of a flow path to pass the fluid through the holes for the sensing.

In this example, the holey fiber itself serves as the flow path, which simplifies the constitution of the flow path and facilitates the production of the sensor.

The change in the properties of the holey fiber to electromagnetic waves depends on the properties and kinds of the substance in the holes of the holey fiber, the properties and kind of the solid portion, the properties and kinds of the material constituting the sensor, temperature and other environmental factors. Therefore the target substance can be detected by detecting the above change. For instance, the transmittance or reflectivity at a certain wavelength of electromagnetic wave in or on a holey fiber filled with a fluid containing no target substance in the holes will change when a target substance-containing fluid is allowed to flow through the holes of the holey fiber. Therefore, the target substance can be detected by detecting the property change by introduction of the target substance into the vacant structure.

FIG. 34 shows a cross-section of holey fiber 1501 employed in this embodiment. Holey fiber 1501 is constituted of a solid portion 1502 and holes 1503. The holes are arranged in a triangle lattice state in a plane perpendicular to the fiber length direction, forming a photonic crystal in the fiber length direction. At the center portion, a hole is eliminated for disturbing the periodicity. This portion serves as a defect in the photonic crystal, and is called a core portion in this photonic crystal fiber.

The photonic crystal having holes 1503 has a photonic band gap, and capable of transmitting the light of the wavelength within this photonic band gap range through the core portion. This light cannot be transmitted in the direction of the plane perpendicular to the fiber length direction owing to the presence of the photonic band gap, and is transmitted through the core portion in the fiber length direction.

However, when the photonic band structure around the core portion is changed by a certain cause and the light wavelength transmitting through the core portion comes outside the shifted photonic band gap range, the light will penetrate the photonic crystal around the core portion and be emitted from the fiber. When a fluid containing a target substance is allowed to flow through the photonic crystal fiber holes carrying a trapping substance, the bonding reaction changes the band structure of the photonic crystal fiber. Therefore, the target substance can be detected by measuring the change in the light intensity emitted from the photonic crystal fiber caused by the bonding reaction.

With a holey fiber other than the photonic crystal fiber, the electromagnetic wave is transmitted by confinement of the electromagnetic wave in the core portion by utilizing total reflection of the electromagnetic wave at the interface between the core portion and the cladding portion. In this case, a portion of the holey fiber is bent, and the electromagnetic wave is projected from the surface of the bent portion at a projection position and a projection angle determined by an alignment means to transmit the electromagnetic wave through the core portion. Herein the “projection position” signifies the region of the holey fiber where the electromagnetic wave is projected, and the “projection angle” herein signifies an angle or direction of projection of the electromagnetic wave measured from a reference direction of a holey fiber.

For detection, another portion of the holey fiber is also bent to leak the electromagnetic wave outside from the surface at the bend portion. If the bonding reaction of the target substance with the trapping substance changes the conditions or force of confinement of the electromagnetic wave in the core portion of the holey fiber, the intensity of the leak of the electromagnetic wave changes at the bent portion of the holey fiber. This change can be utilized for detecting the target substance.

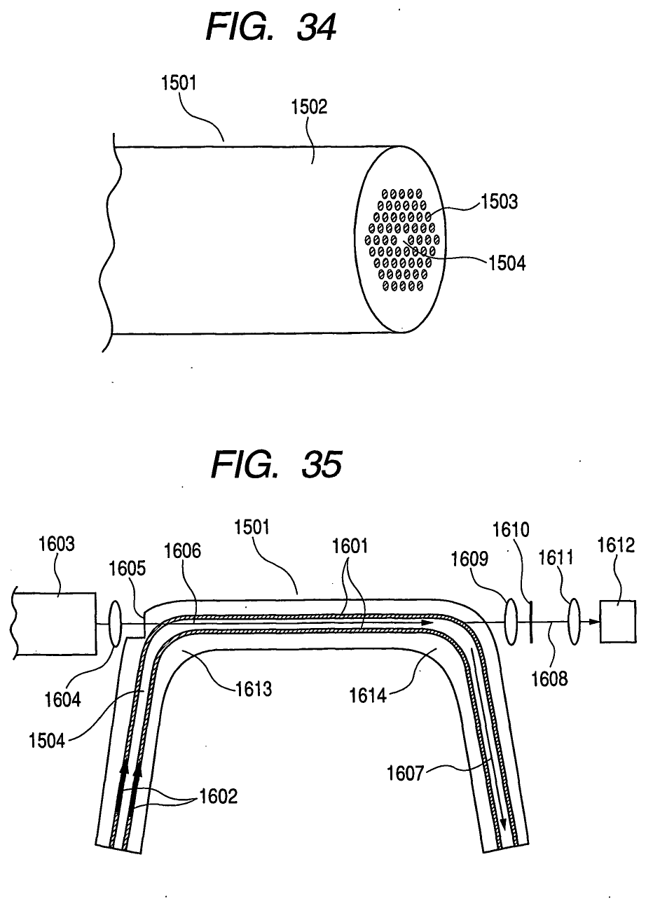

FIG. 35 shows an example of the constitution of the sensor for detecting a target substance by using the photonic crystal fiber 1501 of FIG. 34.

In FIG. 35, the photonic crystal fiber is shown by sectional view along the core portion: the numeral 1504 denotes a core portion, and the numeral 1601 denotes the region where holes are formed. Although cross-sections of two holes are shown in FIG. 35, many holes are provided actually. The numeral 1602 indicates the entire flow direction of the fluid.

The wavelength of light beam 1606 emitted from polarized wave-retaining single mode optical fiber 1603 is selected as below. The selected wavelength is within the photonic band gap region of the photonic crystal fiber when the antibody only is held in the holes of photonic crystal fiber 1501, and the wavelength comes out of the photonic band gap region when the photonic band structure of the photonic crystal fiber is changed by bonding of the antigen to the antibody in the holes of the photonic crystal fiber by the antigen-antibody reaction.

The light beam 1606 emitted from optical fiber 1603 is condensed by the lens and introduced into core portion the photonic crystal fiber. The portion of the photonic crystal fiber for light introduction has bend portion 1613 for facilitating the light introduction, and the light inlet portion 1605 is cut and polished for facilitating the light emission as shown in the drawing. At the detection side of the fiber, bend 1614 is provided to facilitate the emission of the signal light.

Before the fluid containing the antigen is passed through the holes in the photonic crystal, light beam 1606 is confined in core portion 1504 and nearly 100% of the light is transmitted as shown by arrow 1607 along the core portion of the photonic crystal. When the fluid containing the antigen is passed through the holes in the photonic crystal, the antigen-antibody reaction occurs in the holes. With increase of the bonding of the antigen with the antibody in the holes by the antigen-antibody reaction, the confinement of light 1606 in core portion 1504 is weakened, and the intensity of light 1608 as the signal electronic wave is increased. Signal light 1608 is transmitted through lens 1609, polarizing plate 1610, lens 1611, and is detected by photodiode 1612.

The antigen in the fluid can be detected by measurement of the change in the signal light intensity by the antigen-antibody reaction. Plural kinds of the target substances can be detected by use of plural photonic crystal fibers.

The photonic crystal fiber may be replaced by usual holey fiber. The introduction and detection of the electromagnetic wave can be conducted with the holey fiber in the similar manner as with the photonic crystal fiber.

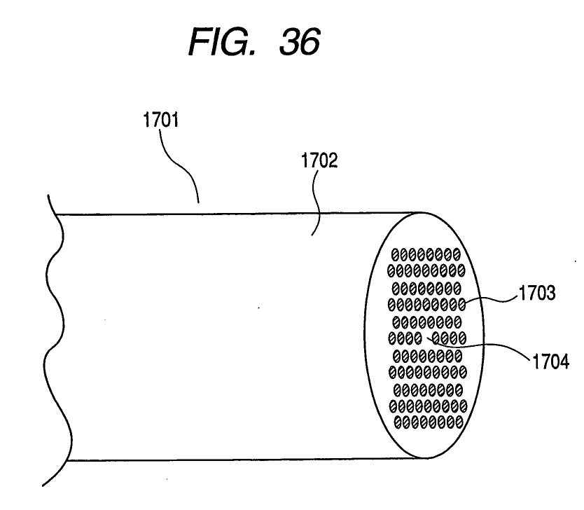

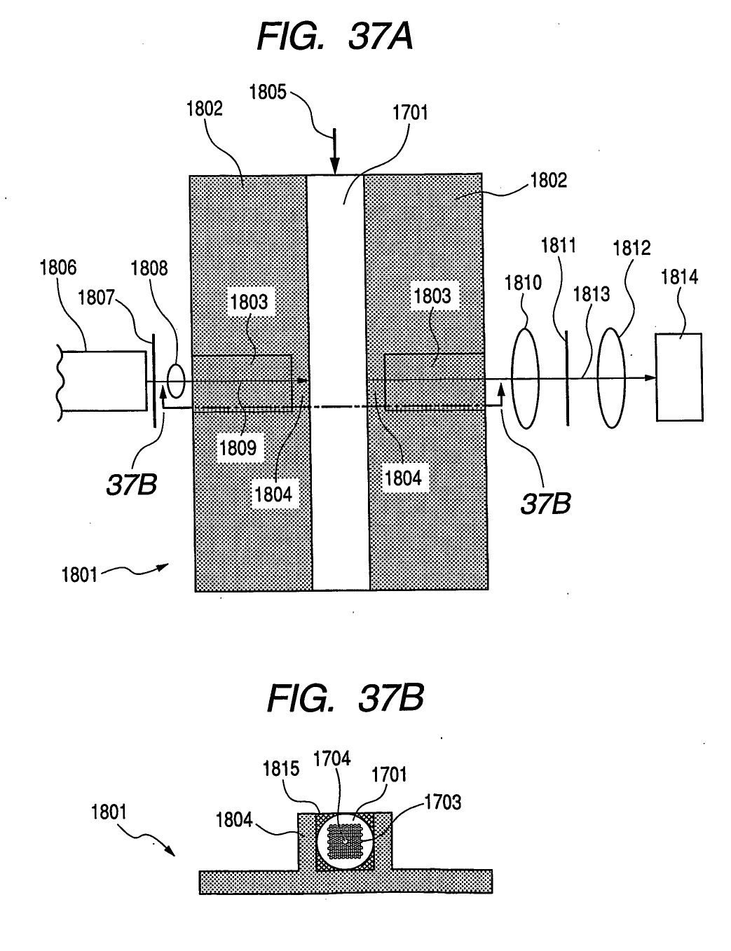

FIGS. 36, 37A and 37B show another example of the sensor employing a photonic crystal fiber.

FIG. 36 shows a cross-section of photonic crystal fiber 1701 employed in this embodiment. Fiber 1701 is constituted of a solid portion 1702 and holes 1703. Holes 1703 are arranged in a triangle lattice in a cross section perpendicular to the fiber length direction, forming a photonic crystal in the fiber length direction. At the center portion, a hole is eliminated for disturbing the periodicity. This portion serves as a defect in the photonic crystal, and is called core portion 1704 in this photonic crystal fiber 1701.

The photonic crystal fiber 1701 has a diameter of 100 μm. The photonic crystal around core portion 1704 has a photonic band gap in the band structure thereof. The core portion can be designed to provide a region capable of transmitting an extremely narrow wavelength range of light, namely a defect level. The core portion of photonic crystal fiber 1701 is designed and prepared as above. The antibody as the trapping agent is preliminarily deposited onto the inside surface of the holes of solid portion 1702 of photonic crystal fiber 1701.

FIGS. 37A and 37B show constitution of a sensor employing photonic crystal fiber 1701 for detecting an antigen as the target substance. FIG. 37B is a sectional view taken along 37B-37B in FIG. 37A.

Sensor chip 1801 is formed by molding. Sensor chip 1801 has a size of about 1.5 mm in length and about 1 mm in width. Photonic crystal fiber 1701 is fit into groove 1815 in the middle portion. The gap between groove 1815 and photonic crystal fiber 1701 are filled with a resin. Portions 1803 of light introduction are formed by partial cutting-off in a width of about 200 μm. Portions 1803 of light introduction are partitioned by barriers 1804 of 10 μm thick from groove 1815.

The wavelength of light beam 1809 emitted from polarized wave-retaining single mode optical fiber 1806 is selected to fall into one of the wavelength ranges corresponding to the defect level of the photonic crystal fiber. The light beam 1809 is controlled by polarizing plate 1807 to obtain TE-polarized light relative to the face of sensor chip 1801, and is introduced into photonic crystal fiber 1701. Signal light 1813 emitted from photonic crystal fiber 1701 is transmitted through lens 1810, polarized plate 1811, and lens 1812, and is detected by photodiode 1814.

A flow of antigen-containing fluid through holes of photonic crystal fiber 1701 causes an antigen-antibody reaction in the holes to change the photonic band structure of the photonic crystal fiber and to change or shift the defect level in the photonic band gap region. The intensity of light 1809 to be projected is adjusted preliminarily before the reaction to correspond to the defect level. By the antigen-antibody reaction, the intensity of the transmitted light 1813, namely signal light, through the photonic crystal fiber is changed. Therefore, the specified kind of antigen as the target substance can be detected by measurement of the change in the signal light intensity by the antigen-antibody reaction.

With the holey fiber, the holes of the holey fiber are utilized as the flow paths. This makes unnecessary to form a photonic crystal in the flow path as described before, whereby the sensor device can be prepared simply and the constitution thereof can be made simpler and smaller.

A flow path portion can be formed by employing a circular groove having a sectional inside diameter equal to the outside diameter of a holey fiber and by fitting the holey fiber into a part of the groove along to the groove. Thereby, the detection portion for detecting the change caused by the bonding reaction and the flow paths for the fluid can be formed.

The holey fiber is constituted of a core portion for light transmission and a cladding portion. With such a constitution for transmission of the electromagnetic wave through the core portion, the transmission path for the electromagnetic wave is confined within the holey fiber, enabling decrease of the size of the entire sensor.

8. Flow Path

The flow path for passing the fluid in the present invention is explained below. The flow path is constituted of a grove or a hole formed on or in a substrate.

The substrate may be made from any material that is resistant against the fluid passed therein. However, at least a portion of the flow path is constituted of a material transmissive to a wave utilized for the sensing. The material of the substrate includes glass materials such as quartz glass, and soda glass; semiconductor materials such as silicon, gallium-arsine; metal materials such as aluminum, and stainless steel; and resin materials such as PMMA (polymethyl methacrylate), COP (cycloolefin polymer), PC (polycarbonate), and acrylic resins.

The breadth and depth of the flow path is not specially limited, provided that the periodic structure of the present invention can be placed in the flow path. The groove or hole as the flow path may be formed by a working method suitable for the substrate material. The working may be conducted by machining such as cutting; injection molding; laser processing; and the like method.

The working can be conducted also by a semiconductor working technique of combination of photolithography and dry etching. For instance, a silicon substrate is worked to form a groove of 100 μm in width and 100 μm in height by a semiconductor process, and in a part of the groove, a periodic structure is placed which has a solid portion and a vacant structure made of the same material as the substrate, silicon, and has a width of 100 μm, a height of 100 μm, and a length of 50 μm in the length direction.

Thus the sensor itself can be miniaturized by employing an electromagnetic wave of a short wavelength and using a periodic structure having cycle period corresponding to the wavelength.

9. Other Constitution of Sensor

The light- or magnetic wave-projecting means of the present invention comprises a laser as the source of light for the sensing. The light from the laser is collimated by a lens or the like for use as the projecting light for sensing. The electromagnetic wave-projecting means in the present invention comprises all the necessary constituting elements for generating an electromagnetic wave suitable of the sensing.

The sensor of the present invention comprises an electromagnetic wave detector for detecting a signal electromagnetic wave. The electromagnetic wave from the electromagnetic wave-projecting means is projected to the periodic structure, and is allowed to penetrate through or is reflected by the periodic structure. The signal electromagnetic wave having penetrated or been reflected is measured by the electromagnetic wave detector. The electromagnetic wave detector is exemplified by a photodiode, and a CCD (charge coupled element) detector. The measured result is transmitted to a computer or the like (not shown in the drawing) to compare the data before and after the antigen-antibody reaction. The difference between the compared data larger than a prescribed difference shows detection of the target substance, and is displayed or recorded.

The sensor of the present invention comprises a wave polarization-controlling means for controlling polarization of the projected electromagnetic wave. In the present invention, the wave polarization signifies spatial polarization of electromagnetic field of the electromagnetic wave. For instance, in the case where the electromagnetic wave is a light beam of visible light, infrared light, or ultraviolet light, the wave polarization is light polarization. For instance, for the light beam as the electromagnetic wave beam, the wave polarization-controlling means includes optical polarizers, a Glan-Taler prisms, and λ1/2λ planes. Some of the fibers or photonic crystals employed in the sensor of the present invention have polarization-dependency of the electromagnetic wave according to the structure. Two-dimensional photonic crystals have remarkable polarization-dependency owing to structural anisotropy. The polarization dependency on the electromagnetic wave enables precise high-sensitivity detection of the target substance by polarizing the projecting magnetic wave to be suitable for the sensing by using the wave polarization-controlling means.

The sensor of the present invention may comprise an alignment means for controlling the direction, angle, position and so forth of the projected electromagnetic wave to detect the signal electromagnetic wave effectively. The position of the projection signifies the site of the photonic crystal or holey fiber where the electromagnetic wave is projected. The angle of projection signifies an angle of the projected electromagnetic wave from a prescribed direction of the periodic structure or the holey fiber.

For instance, consider the case where an electromagnetic wave is projected onto a glass holey fiber of 1 mm long carrying a trapping agent at a region of an equal distance, 0.5 mm, from the both ends where a larger amount of the trapping substance is supported, and the reflected electromagnetic wave is detected as the signal electromagnetic wave. In this case, the aligning means controls projection to direct to the position at equal distances, 0.5 mm each, from the both ends at an angle, for instance 30°, from a prescribed reference direction, for instance the holey fiber length direction, to maximize the intensity of signal electromagnetic light. Thereby, the target substance can be detected effectively and efficiently by increasing the intensity of the signal electromagnetic wave to be detected and making clear the properties such as the wave length thereof.

The sensor of the present invention may comprise a temperature controlling means for stabilizing the performance of the sensor and conducts the sensing stably at a high sensitivity. In this Specification, the temperature-controlling means can control the temperature of any of the photonic crystal, holey fiber, and photonic crystal fiber, or the flow path. For instance, the photonic crystal is placed on a Peltier element and the Peltier element is controlled by feed-back to control the temperature variation of the periodic structure. In this instance, the Peltier element and the controller are both included in the temperature-controlling means.

10. Detection Object

In the present invention the fluid as the object of the detection is a gas or liquid, but the fluid may contain a solid component insofar as it has fluidity.

The target substance to be detected by the sensor is mixed or dissolved in the above gaseous or liquid fluid. The sensor for detecting a target substance in a gas includes a gas sensor for detecting carbon monoxide in automotive exhaust gas; and a sensor for detecting dusts, sulfur oxides, and the like which affect working environment in factories and offices. The sensors for detecting a target substance in a liquid fluid are roughly classified into the groups below:

(1) Sensors for environmental pollutant,

(2) Sensors for process and quality control, combinatorial synthesis, and combinatorial screening, and

(3) Sensors for diagnosis of disease and health conditions.

The sensors of classification (1) include sensors for water quality of rivers, lakes, and sea water; sensors for analysis of agricultural chemicals, and environmental hormone in waste water of agriculture and forestry. For detecting an environmental pollutant as the target substance in soil or a solid waste, the environmental pollutant is extracted from the soil or the solid waste with a liquid medium and, if necessary, the solid component is removed by filtration or a like procedure to obtain a liquid fluid for detection by the sensor of the present invention.

The sensors of classification (2) include sensors for food analysis and sensors for combinatorial synthesis and for combinatorial screening in chemical industries and pharmaceutical industries. In particular, in recent years in drug design in pharmaceutical industry, interactions between proteins or between peptides are being elucidated and the results are coming to be applied to protein preparations and gene preparations. A target substance having a useful pharmacological effect can be screened by immobilizing a specified DNA or protein as the trapping substance to the solid portion of the present invention and by detecting a DNA, protein, peptide or chemical synthesized compound from enormous library.

The sensors of classification (3) include sensors for detecting, as a target substance, a protein, sugar protein, lipoprotein, peptide or a composite thereof which are called a disease marker contained in a body liquid such as blood of a testee or contained in a liquid extract solution obtained from a diseased part. Such a target substance is detected by introduction into the flow path having a trapping substance specific to the target substance to cause a specific bonding reaction. In another system, a part of gene relating to a specific disease in a single strand state is employed as the trapping substance (sometimes called a DNA probe), and a specimen extracted from the cell of a testee is introduced into the flow path to cause complementary specific bonding of the genes for the detection.

11. Target Substance and Trapping Substance

A trapping substance which is capable of bonding to the target substance is immobilized on the surface of the photonic crystal structure.

The target substance especially useful as the object of detection of the sensor in the present invention includes substances useful physiologically, and exists in many cases in the specimen, as a mixture with other substances or at a low concentration in a wide distribution range. Therefore, a means and method for detecting only the target substance selectively is demanded.

For the above purpose, in the method using the sensor of the present invention, “a substance having affinity to bond to the target substance” capable of trapping the target substance only (hereinafter occasionally called a “trapping substance”) is employed.

The “target substance” to be detected by the present invention and the “molecule having affinity to bond to the target substance (trapping substance)” include specifically nucleic acids, proteins, peptides, sugar chains, lipids, and lower molecular compounds, and composites thereof, and substances containing a part of the aforementioned molecule. When the protein is an immunoreaction product, antibodies, antigens, haptens, and complexes thereof are useful.

For an antibody as the target substance, the trapping substance is an antigen capable of bonding to the antibody.

The sensor of the present invention is applicable in the case where the target substance is a DNA and the trapping substance is a DNA or oligonucleotide complementary to the target DNA. The trapping substance may be directly bonded to the photonic crystal structure material, or may be bonded indirectly with interposition of a supporting substance which is capable of bonding both to the trapping substance and to the photonic crystal structure material to bond the trapping substance substantially to the constituting material.

The combination of the trapping substance and the target substance includes, in addition to the aforementioned combinations of an antigen and an antibody, and hybridization of DNAS, combinations of a protein with a lower molecular compound, such as an enzyme with a substrate thereof, a hormone and a receptor therefor, and avdin or streptoavidin with biotin; combinations of a protein and a sugar chain such as lectin (concanavalin A) and cello-oligomer; and combinations of a protein like a transcription factor and a nucleic acid DNA. In particular, in the combination of an antibody and a low molecular compound, the low molecular compound is called a hapten and is used usually in combination with an assisting protein. In a usual antigen-antibody reaction, when the antibody is the target substance, the antibody may be preliminarily bonded to a secondary antibody before the sensing for improving the sensing effect of the present invention. For the same purpose, a metal colloid or semiconductor quantum dot particles may be bonded to the target compound by considering the vacant volume of the sensor of the present invention.

In the sensor of the present invention, the “bonding” of the target substance to the trapping substance signifies specific chemical or physical bonding between the two molecules to form a bonding pair.

In addition to the bonding of antigen-antibody by a known antigen-antibody reaction, the bonding includes combinations such as biotin and avidin; a carbohydrate and lectin; complementary sequences of nucleic acid-nucleotide; an effector and a receptor; a cofactor and an enzyme; an enzyme inhibitor and an enzyme; a peptide sequence and a specific antibody to the sequence or the entire protein; a polymer acid and a base; a dye and a protein binder; a peptide and a specific protein binder (ribonuclease, S-peptide, and ribonuclease S-protein); a sugar and boric acid; and bonding between a molecular pair having affinity enabling molecular association in bonding assay; but are not limited thereto.

The bonding pair further includes factors of analogue of the original bonding element such as an analogue of the target substance prepared by a recombination technique or molecular engineering.

An immune reactant as the bonding element may be an antibody, an antigen, a hapten, or a complex thereof. The “antibody” includes monochronal antibodies, polychronal antibodies, recombinant protein type antibodies, natural type antibodies, chimeric antibody, mixtures thereof, single chain antibody-exhibiting phage antibody (including entire phage), a fragment or fragments exhibiting the single chain antibody, and mixtures of bonding elements of an antibody and a protein.

Recently, with development of evolutional molecular engineering, a technique of screening of an aptamer (sometimes called a nucleic acid antibody) has been developed (systematic evolution of ligands by exponential enrichment; SELEX or in vitro selection). This technique serves to screen a nucleic acid molecule having high affinity to a target molecule such as a protein from a random oligonucleotid-library.

Many papers have been presented on production of a high affinity ligand by screening with the above aptamer more rapidly and more easily than with the antibody (e.g., Nature, 355:564 (1992); International Patent Application WO92/14843; Japanese Patent Application Lai-Open Nos. 8-252100, 9-216895; etc.).

Further, the bonding of a transcription factor of a nucleic acid, a protein, to a nucleic acid having a specified base sequence can be useful in research of causes of diseases, and effective diagnosis and treatment of the diseases.

The “bonding” which is the object of the sensor of the present invention includes naturally affinitive bonding between a nucleic acid and a protein.

The “bonding” in the sensor of the present invention may be permanent or temporary and includes any physical adhesion and chemical adhesion, and close specific selective association. Generally, the objective ligand molecule can be allowed to adhere to a receptor physically by interaction of ionic bonding, hydrogen bonding, hydrophobic force, Van der Waal's force, or the like. The interaction of “bonding” can be short like in the bonding causing a chemical reaction. This occurs generally when a bonding component is an enzyme and the analysis object “bonding substance” is the substrate for the enzyme. Further the chemical linkage can be permanent or reversible. The bonding can become specific by change in the conditions.

In the present invention, the target substance is brought into contact with the trapping agent immobilized on the surface of the solid portion usually in an aqueous medium. In the case where the target substance is slightly soluble in water like a possible drug substance, a polar solvent such as alcohol, acetone, DMSO (dimethylsulfoxide), and DMF (dimethylformamide), or a surfactant such as Tween, Triton, and SDS is added thereto, or a nonpolar solvent is added and the contact is conducted in an emulsion system to accelerate the bonding reaction.

In the case where such a solvent or surfactant is employed as the additive, the concentration of the additive should be in the range not to impair the affinity bonding performance of the immobilized trapping substance.

For promoting the contact and bonding of the target substance to the trapping substance immobilized on the surface of the solid part in the method of the present invention, a heating means or a stirring means such as ultrasonic means may be employed provided that the affinity bonding function of the trapping substance is not impaired.

For preventing non-specific adsorption on the solid surface, the surface of the solid portion not immobilizing the trapping substance is preferably coated with a “blocking agent” which does not impair the activity of the trapping agent. The blocking agent for the blocking treatment includes phospholipid polymers, collagen, gelatin (especially cold-water fish skin gelatin), skimmed milk, serum protein like BSA, and many compounds having a hydrophobic portion or hydrophilic portion not reactive to a protein.

12. Immobilization of Trapping Substance

The molecule (trapping substance) having bonding affinity to the target substance can be immobilized on the surface of vacant structure of the solid portion of the sensor by physical adsorption by physical affinity such as hydrophobicity, ionic attraction, Van der Waals force, or the like. However, in consideration of reproducibility and stability of the immobilization, preferably, the solid portion is treated with a surface modifier having a functional group, and then the functional group is allowed to react with the functional group of the trapping substance in presence or absence of a converting, modifying, or activating reagent to form irreversible covalent bond.

Otherwise, utilizing the above functional group, a substance capable of bonding to the “trapping agent” to be immobilized on the surface of the solid portion (e.g., protein A, protein G, etc. for an antibody as the trapping agent) is bonded onto the surface, and the trapping substance is specifically bonded thereto. The trapping agent may be modified and then immobilized in the same manner as above. In an example of the latter method, on the surface of the carboxyl type structure, biotin is immobilized by use of a reagent NHS-iminobiotin (Pierce Co.), and on the other hand, a trapping substance having been modified by avidin or streptavidin is specifically bonded to the above biotin.

In another method, the structure on which a trapping substance is to be immobilized is brought into contact with a biological sample liquid such as a tissue or cell homogenate, or serum containing a natural protein to allow the natural protein to adsorb or bond to the trapping substance to be immobilized on the structure surface.

In still another method, as practiced in the known phage-display antibody selection method, the antibody portion displayed on the surface of a suitable bacteriophage is bonded selectively to the magnetic structure to which a protein as the trapping substance is to be immobilized.

In still another method, the structure is brought into contact with a biological sample containing a receptor such as a hybridoma supernatant liquid, and phage display, and the receptor contained in the biological sample is allowed to adsorb specifically immobilized onto the structure.

The sensor of the present invention is capable of detecting plural target substances concurrently as one of the feature. However, preliminary separation of the target substances in the fluid can be effective in some cases. When the sample is gaseous, generally the separation can be conducted by gas chromatography, and when the sample is liquid, the separation is conducted generally by liquid chromatography and electrophoresis. However the means are not limited thereto insofar as the target substances in the fluid can be separated.

In the sensor of the present invention, the target substance need not be labeled by a labeling agent such as a fluorescent substance, fine metal particles, fine semiconductor particles, and an enzyme. However, in the cases when refractive index change is not large, or when the target substance has much lower molecular weight, or the like cases, the target substance can be labeled with the labeling agent.

### EXAMPLES

### Example 1

An example of a sensor device is shown below which employs the aforementioned photonic crystal in combination with a light source as the light-projecting means, and detector as the means for detecting the emitted light.

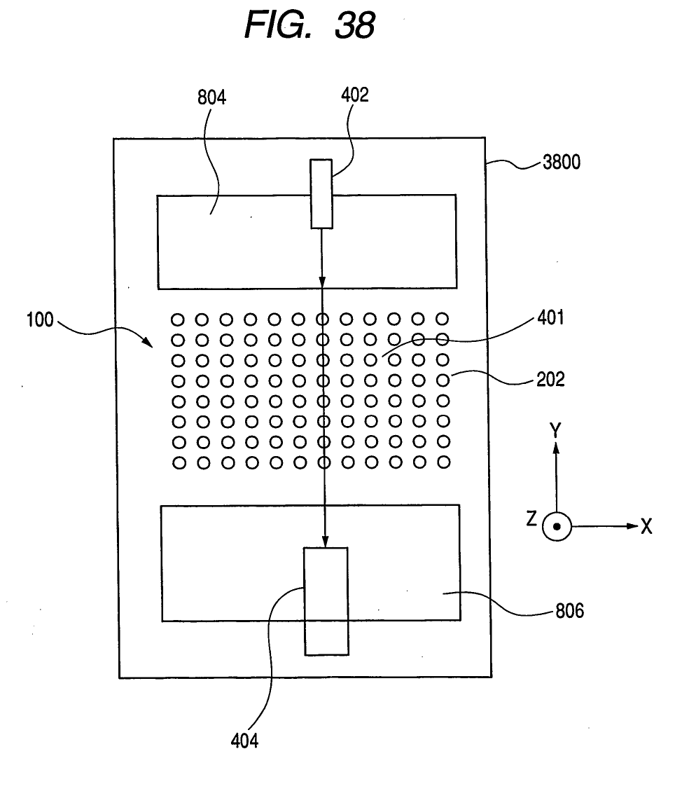

FIG. 38 shows a first example of the sensor of the present invention. The numeral 3800 denotes a main body package of a biosensor, and the numeral 100 denotes a photonic crystal structure. The photonic crystal structure in FIG. 38 has holes 202 arranged periodically in a solid. A test sample liquid is passed through the holes. A trapping substance is held on the surface of the holes. A light beam from light source 402 of a laser or the like is collimated and is projected to photonic crystal structure 100. The light having been transmitted through the photonic crystal is introduced to signal light detector 404. The change in photonic band gap energy is detected by signal light detector 404. The presence or absence of the target substance is detected from the change. The detection is conducted in a manner described above. In FIG. 38, the projected light beam is shown to be projected through cavity 804 to photonic crystal structure 100 and the light beam emitted from the photonic crystal is shown to be introduced through cavity 806 to signal light detector 404. However, the cavity is not necessary and the cavity portion may be constituted of a material transparent to the light employed.

Naturally the photonic crystal structure may be a two-dimensional photonic crystal having periodic columnar structure as shown in FIG. 3 or FIG. 8.

The detection may be conducted during flow of the test sample liquid through the vacant structure, or after completion of the flow of the test sample liquid. Otherwise the detection may be conducted with the vacant structure filled another liquid after the completion of the flow of the test sample liquid, or after evaporation of the test sample liquid.

The quantity of the target substance can be derived precisely by calculation from the output of the detector by using a calculation unit provided for calculation by a prescribed calculation process. For instance, in the case where the transmitted light intensity is changed by adhesion of the target substance, a reference table is prepared which shows the transmitted light intensity as a function of the quantity or concentration of the target substance contained in the test sample liquid, and the calculation is conducted on the basis of the reference table.

Such a biosensor need not be packaged in one unit including all of the parts. The light-emitting units and the light-receiving unit may be separated from the package containing the photonic crystal. With this separate constitution, the light-receiving unit and the light-emitting unit can be used repeatedly to lower the device cost advantageously.

### Example 2

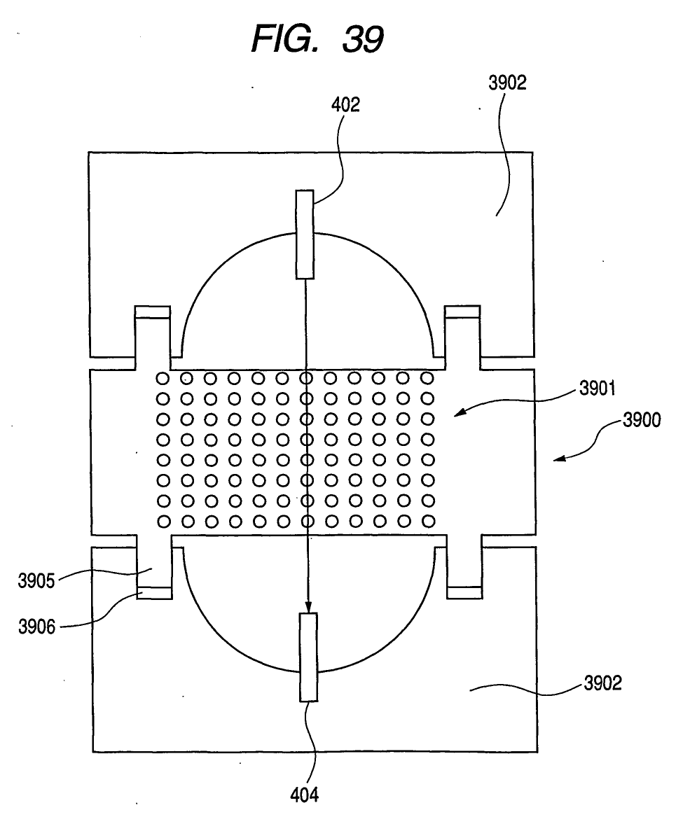

FIG. 39 shows a second example of the sensor of the present invention. Biosensor unit 3900 has photonic crystal 3901 containing a reactive substance. External units 3902 contain light-emitting means 402 and signal light-detecting means 404. Biosensor 3900 having the photonic crystal is connected thereto.

The sensor of this example is equipped with an aligning mechanism which serves to introduce the light beam generated by the light source in the external unit into the photonic crystal and serves to introduce the light beam emitted from the sensor unit into a light detecting means in the external unit. The aligning mechanism specifically may be a simple one having a protrusion and a depression for coupling. An example is shown in FIG. 39 which has protrusion 3905 provided on biosensor unit 3900 and a depression 3906 provided on external unit 3902. Preferably the aligning mechanism has further a function for adjusting the light introduction position on the photonic crystal and the light detection position for detecting the light emitted from the photonic crystal after coupling of the protrusion and the depression of the alignment mechanism.

With the detection system of this example constructed of biosensor unit 3900 and external units 3902, the biosensor unit can be changed for each of the specimens, and the same light emitting means and the same light receiving means can be commonly repeatedly used to lower the cost. For the same reason, the calculation unit may be mounted on the external unit.

### Example 3

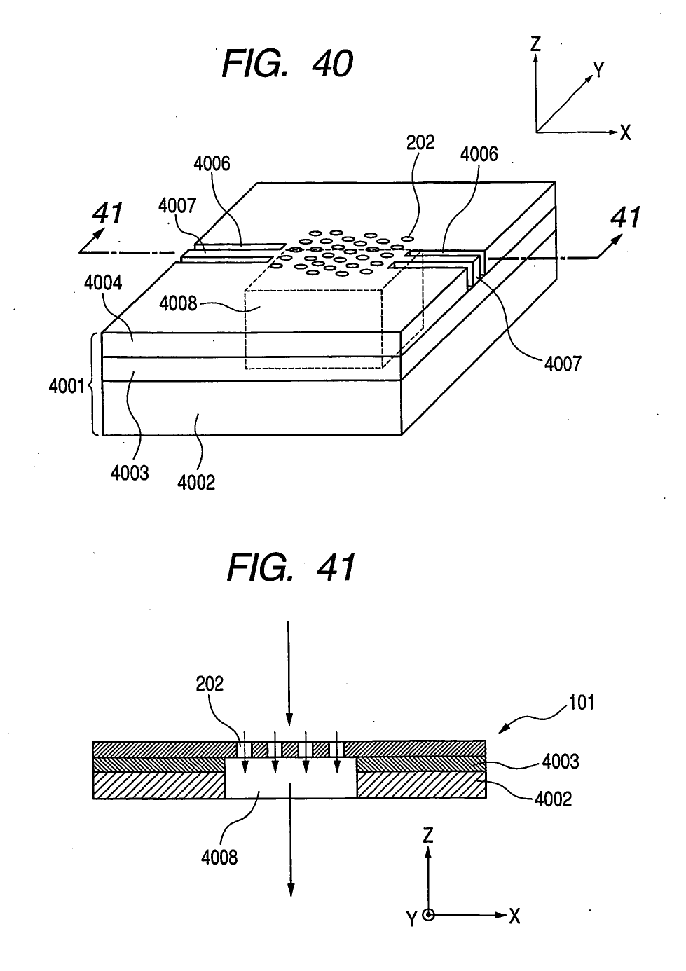

FIGS. 40 and 41 show an example of the sensor of the present invention. FIG. 40 is a perspective view of the sensor. FIG. 41 is a sectional view taken at a plane 41-41 parallel to the ZX plane including optical waveguide 4007. SOI (Silicon on Insulator) substrate 4001 is a thin film constituted of base plate 4002, insulating layer 4003 of about 1 μm thick, and SOI layer 4004 of about 200 nm. In SOI layer 4004, there are formed grooves 4006 of about 1 μm wide to provide two optical waveguides 4007 therebetween, and holes of about 110 nm in radius bored in the layer thickness direction arranged in a two-dimensional triangle lattice at a lattice constant of about 400 nm in the region of 100 μm×100 μm between the two optical waveguide 4007 by electron ray lithography and dry etching, or a like process. The periodic structure having plural holes arranged periodically functions as a photonic crystal. The insulating layer is also called a BOX layer (Barried Oxide Layer). A part of insulating layer 4003 and base plate 4002 in the region under the photonic crystal formation is removed from back side by dry-etching to provide vacant region 4008. The holes 202 of the photonic crystal are connected spatially to vacant region 4008. Thereby SOI substrate 4001 is pierced in the direction perpendicular to the two-dimensional plane. Vacant region 4008 serves as a part of the flow path. Thereby, the fluid containing the target substance can be passed, as shown by the arrow in FIG. 41, through holes 202 of SOI layer 4004 and vacant region 4008. An antigen capable of selectively adsorbing an antibody as the target substance is fixed on the side walls of the holes. A light beam to be utilized for the detection is introduced from one side of the two optical waveguides 4007. The light emitted from the other optical waveguide is subjected to measurement. The target substance can be detected by difference of the spectrum of the measured light before and after the adsorption of the antibody by the antigen.

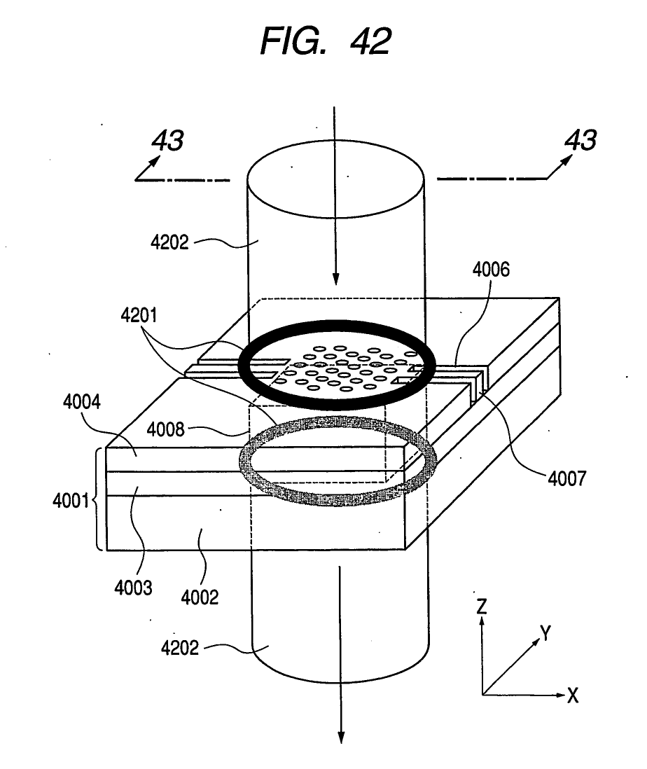

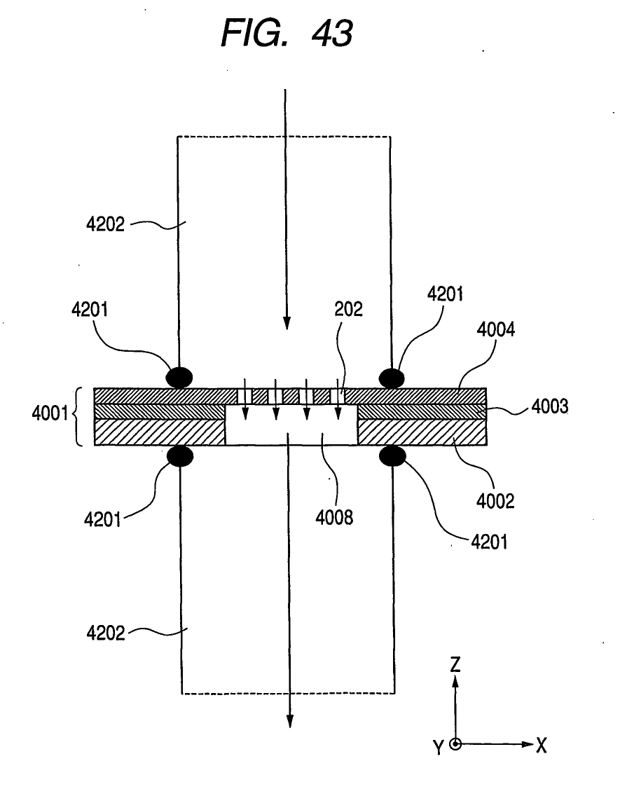

FIGS. 42 and 43 shows a constitution in which flow paths 4202 and O-ring 4201 for prevention of fluid leakage are added to the constitution of FIGS. 40 and 41. FIG. 42 is a perspective view. FIG. 43 is a sectional view taken along plane 43-43 parallel to the ZX plane including optical waveguide 4007. With this constitution the fluid containing the target substance can be passed through the region of the photonic crystal and vacant region 4008 locally for efficient detection.

### Example 4

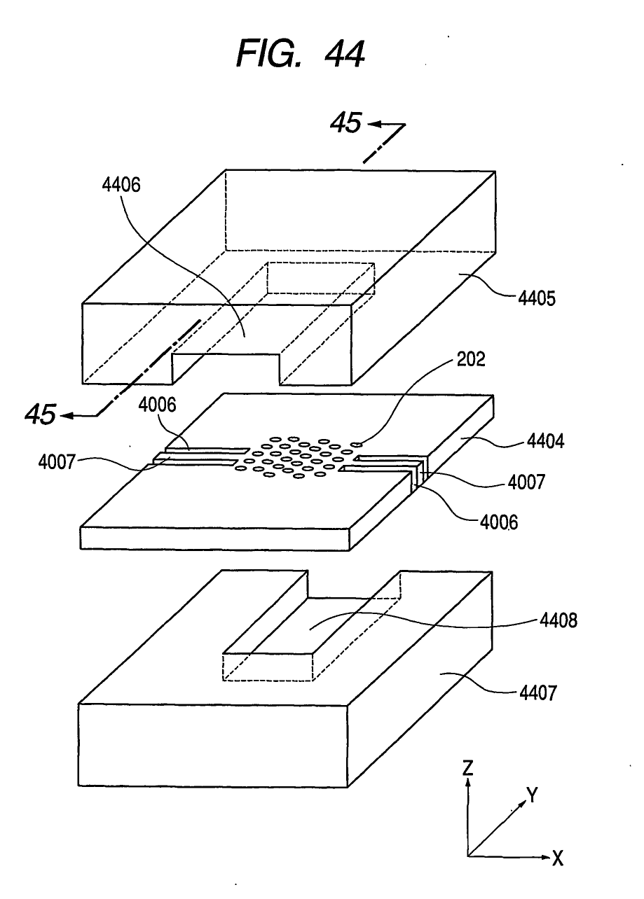

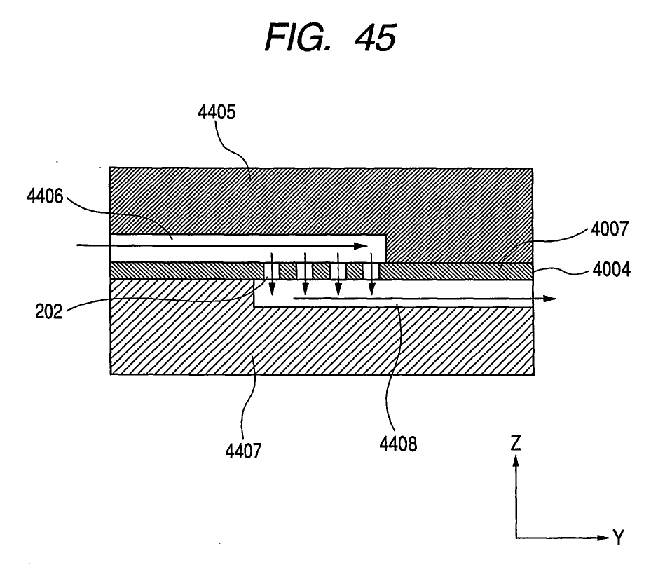

FIGS. 44 and 45 shows still another example of the sensor of the present invention. FIG. 44 is a perspective view of the sensor. FIG. 45 is a sectional view taken at a plane 45-45 parallel to the YZ plane including the photonic crystal region. The numeral 4004 denotes a thin film of about 1 μm thick. This thin film has two optical waveguides 4007 of about 5 μm wide formed by grooves 4006 of about 1 μm wide on the both sides thereof, and a photonic crystal region having holes 202 of about 110 nm in radius bored in layer thickness direction and arranged in a periodic triangle lattice structure of a lattice constant of about 400 nm. Grooves 4006 and holes 202 penetrate though the thin film perpendicularly to the thin film face. Flow path members 4405,4407 is prepared by working a flat plate of a PDM material to form vacant regions 4406 and 4408 as the flow paths. Thin film 4004 is held between flow path member 4405,4407 to form a laminate. Thereby a sensor is constructed which has an integrated flow path. In place of the PDMS material, Si, SiO₂, or the like may be used. In this constitution, vacant regions 4406,4408 as the flow paths in flow path member 4405,4407 overlap partly with the photonic crystal region in the direction perpendicular to the face of thin film 4004. With this constitution, the fluid can flow from flow path 4406 through plural holes 202 to flow path 4408 without leakage of the fluid as shown by the arrow in FIG. 45.

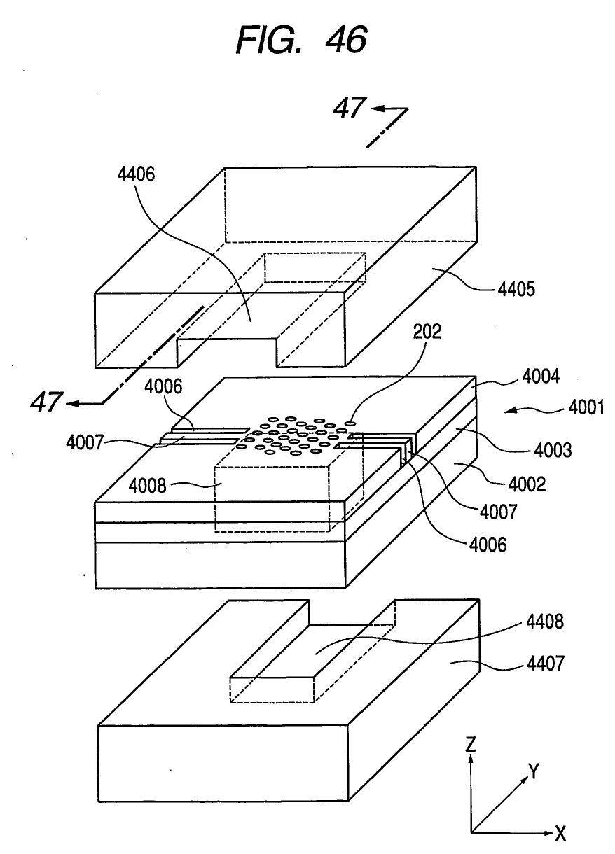

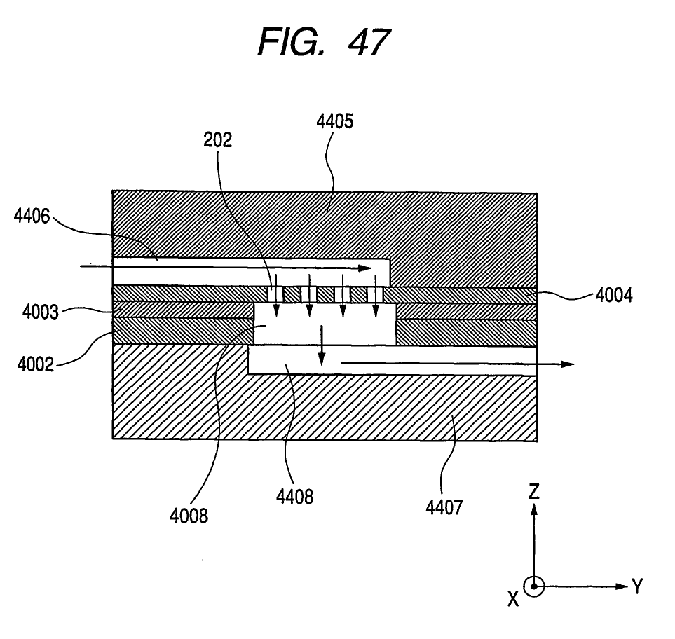

FIGS. 46 and 47 show another construction of the sensor in this example in which SOI substrate 4001 is used in place of thin film 4004. FIG. 46 is a perspective view of the sensor. FIG. 47 is a sectional view taken at a plane 47-47 parallel to the ZX plane including the photonic crystal region. SOI substrate 4001 is constituted of base plate portion 4002, insulating layer 4003 of about 1 μm thick, and SOI layer 4004 of about 200 nm thick. SOI substrate 4001 is produced by forming, on SOI layer 4004, grooves 4006 of about 1 μm wide to provide optical waveguides 4007 therebetween, and holes 202 of about 110 nm in radius bored in the layer thickness direction arranged in a two-dimensional triangle lattice at a lattice constant of about 400 nm in the region of 100 μm×100 μm between the two optical waveguide 4007 by electron ray lithography and dry etching, or a like process. The periodic structure having plural holes 202 arranged periodically functions as a photonic crystal. A part of insulating layer 4003 and base plate 4002 in the region under the photonic crystal region is removed from back side of the base plate by dry-etching to provide vacant region 4008. Holes 202 of the photonic crystal are connected spatially to vacant region 4008. Thereby SOI substrate 4001 is pierced in the direction perpendicular to the two-dimensional plane. In producing such a construction, the insulating layer may be etched by HF (hydrofluoric acid) to form the vacant region. Vacant region 4008 serves as a part of the flow path. With this construction, the fluid containing the target substance can be passed from flow path 4406 through plural holes 202 and vacant region 4008 to flow path 4408. Flow path member 4405, 4407 and SOI substrate 4001 are laminated in superposition such that a part of flow path 4406 overlaps with the photonic crystal portion but does not overlap with groove 4006, and flow path 4408 overlaps with vacant region 4008. Thereby the fluid containing the target substance is allowed to flow from flow path 4406 to flow path 4408 without leakage.

The target substance is detected by utilizing the light transmitted through the optical waveguide in the same manner as in Example 3.

### Example 5

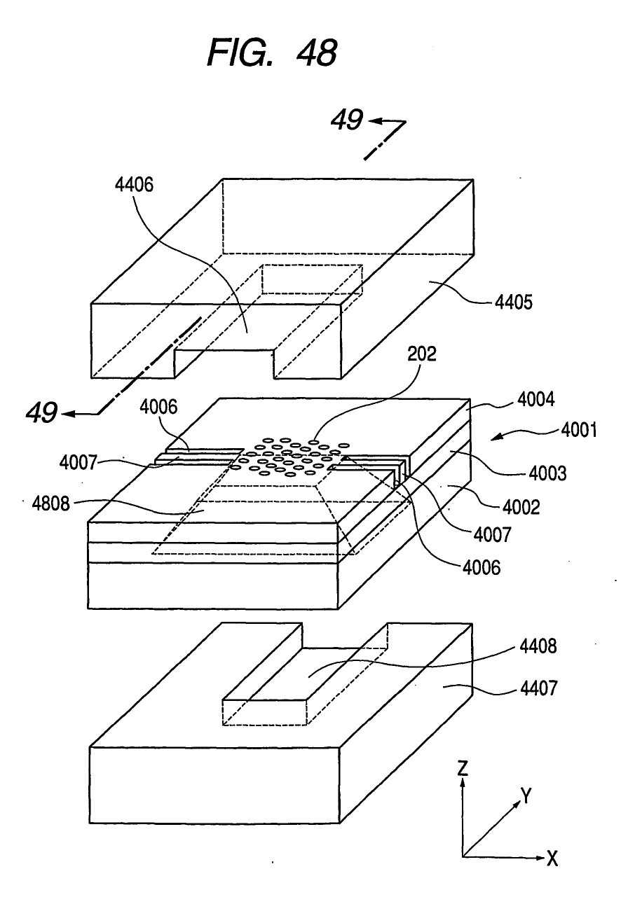

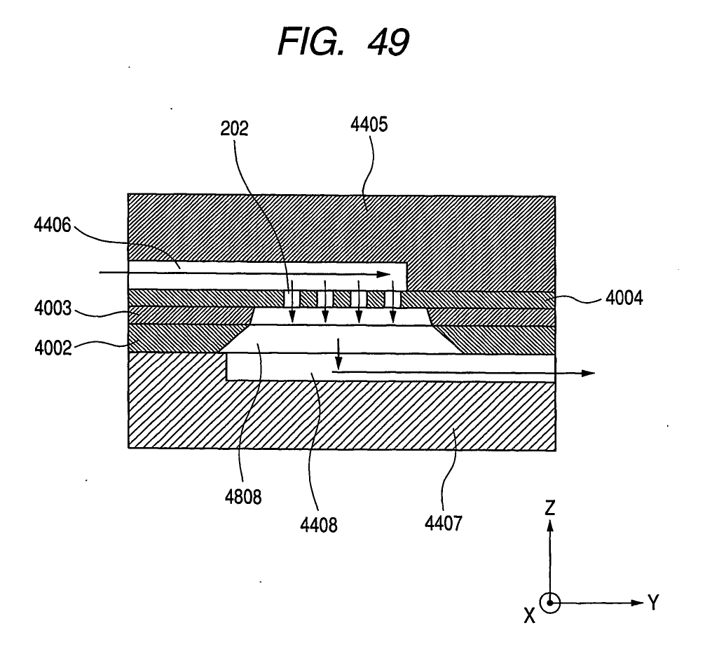

FIGS. 48 and 49 show still another example of the sensor of the present invention. This sensor is produced by forming vacant region 4008 by anisotropic dry etching of insulating layer 4003 and base plate 4002 constituting SOI substrate 4001 in Example 4. In this example, a part of vacant region 4408 as the flow path in flow path member 4407 overlaps with vacant region 4808 of SOI substrate 4001 in the direction perpendicular to the face of SOI substrate 4001. FIG. 48 is a perspective view of the sensor of this example. FIG. 49 is a sectional view taken at a plane 49-49 parallel to the YZ plane including the photonic crystal region.

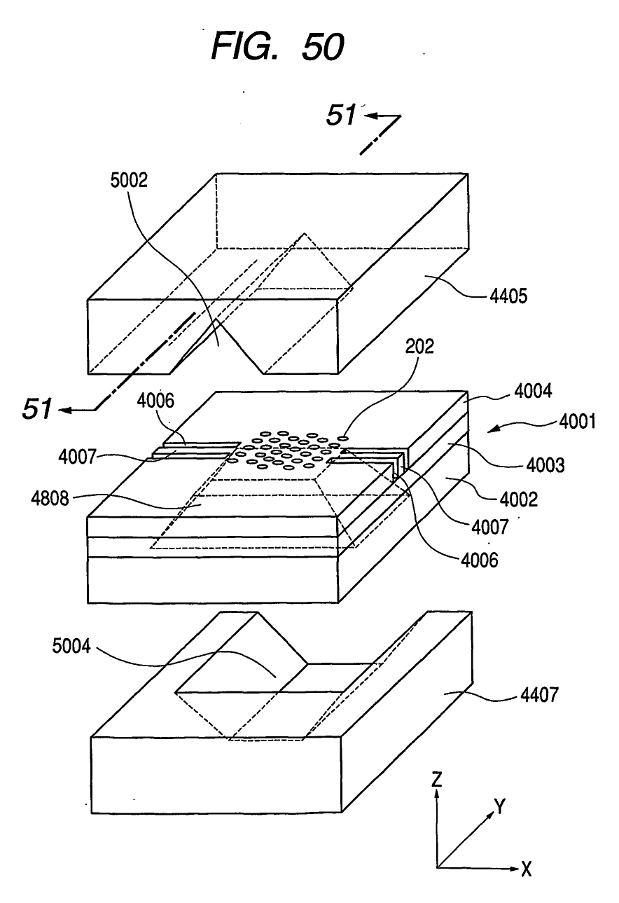

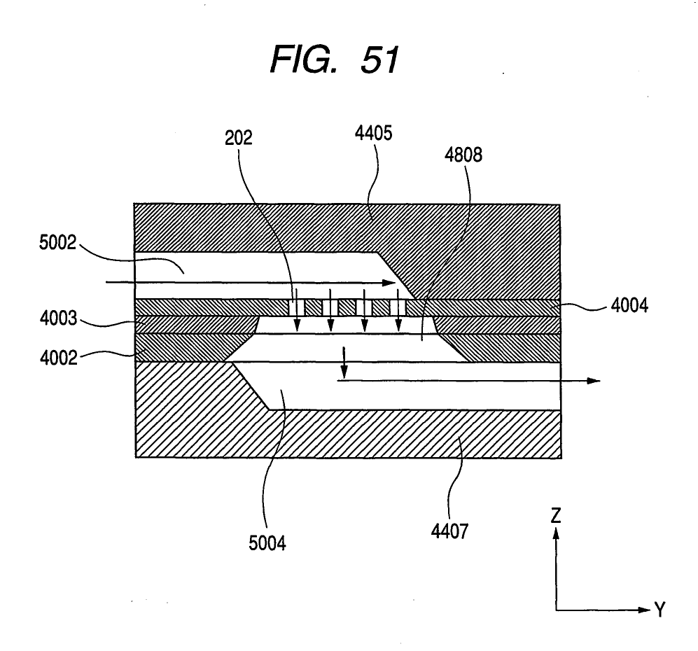

Further, as shown in FIGS. 50 and 51, flow path members 4405,4407 may have flow paths 5002,5004 formed by anisotropic etching by employing KOHTMAH. FIG. 50 is a perspective view of this sensor. FIG. 51 is a sectional view taken at a plane parallel to the YZ plane including the photonic crystal region. For formation of vacant regions 4808,5002,5004 having a slanting face, for example, the (100) plane is utilized as the plane parallel to the face of SOI substrate 4001, and the slanting face is formed by {111} plane.

The target substance is detected by utilizing the light transmitted through the optical waveguide in the same manner as in Example 3.

### Example 6

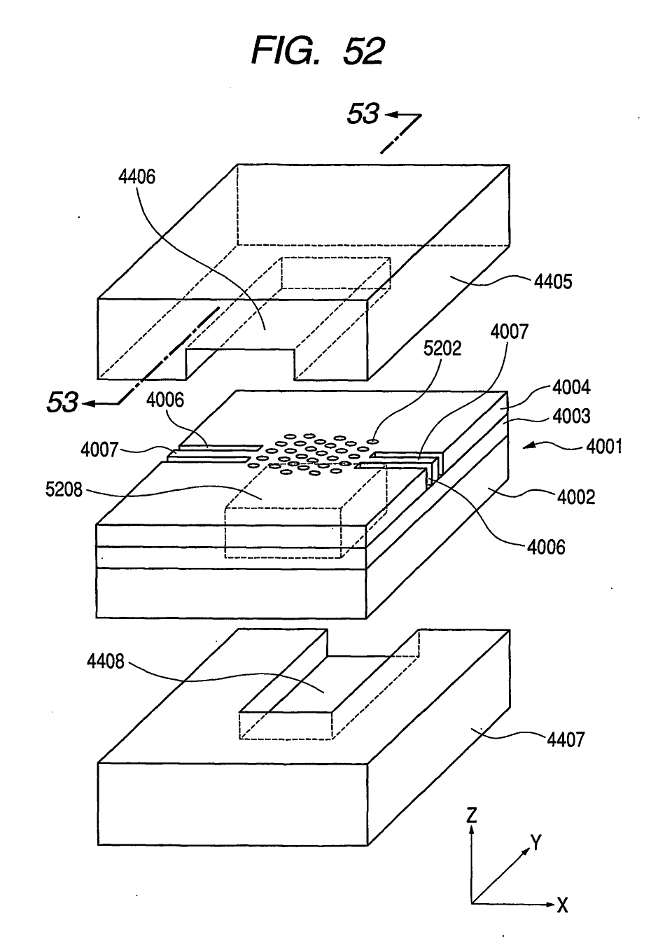

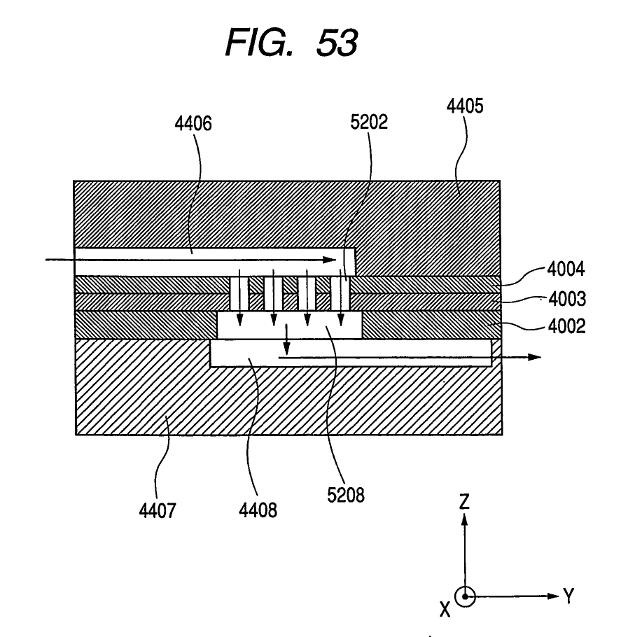

FIGS. 52 and 53 show an example of the sensor of the present invention. FIG. 52 is a perspective view of the sensor. FIG. 53 is a sectional view taken at a plane 53-53 parallel to the YZ plane including photonic crystal region. SOI substrate 4001 is constituted of base plate member 4002, insulating layer 4003 of about 1 μm thick formed from SiO₂, and SOI layer 4004 of about 220 nm thick. In SOI layer 4004, there are formed grooves 4006 of about 1 μm wide to provide optical waveguides 4007 therebetween, and holes of about 110 nm in radius bored in the layer thickness direction arranged in a two-dimensional triangle lattice at a lattice constant of about 400 nm in the region of 100 μm×100 μm between the two optical waveguide 4007 by electron ray lithography and dry etching, or a like process. The periodic structure having periodically arranged plural holes functions as a photonic crystal. A part of base plate member 4002 in the region in superposition on the photonic crystal formation in the direction perpendicular to the face of SOI substrate 4001 is removed from back side by dry-etching to provide vacant region 5208. The holes 5202 of the photonic crystal are connected spatially to vacant region 5208. Thereby SOI substrate 4001 is pierced in the direction perpendicular to the two-dimensional plane. Vacant region 5208 serves as a part of the flow path. Thereby, the fluid containing the target substance can be passed through holes 5202 formed through SOI layer 4004 and insulating layer 4003 and vacant region 5208 as shown by the arrows in FIG. 53. Flow path members 4405,4407 are superposed in lamination with interposition of SOI layer 4404. In lamination of flow path members 4405,4407 and SOI substrate 4001, a part of flow path 4406 overlaps with the photonic crystal region in the direction perpendicular to the substrate face, but does not overlap with groove 4007. Thereby the fluid containing the target substance is allowed to flow from flow path 4406 through holes 5202 and vacant region 5208 to flow path 4408.

The target substance is detected by utilizing the light transmitted through the optical waveguide in the same manner as in Example 3.

This application claims priority from Japanese Patent Application No. 2003-303115 filed Aug. 27, 2003, No. 2003-302520 filed Aug. 27, 2003, and No. 2004-247468 filed Aug. 26, 2004, which is hereby incorporated by reference herein.

1. A device for detecting a target substance in a fluid, comprising
a periodic structure having a vacant portion for passing a fluid containing the target substance and a solid portion capable of transmitting an electromagnetic wave arranged regularly to form a periodic distribution of a refractive index for the electromagnetic wave,
an electromagnetic wave-projecting means for projecting the electromagnetic wave to the periodic structure, and
a detecting means for measuring the magnetic wave emitted from the periodic structure to detect a change in the periodic distribution of the refractive index.

2. The device according claim 1, wherein a trapping substance capable of bonding selectively to the target substance is disposed on the surface of the solid portion, and a change in the periodic distribution of the refractive index caused by bonding the target substance to the trapping substance is detected.

3. The device according to claim 1, wherein the periodic structure forbids transmission of the electromagnetic wave in a specific wavelength band depending on the periodic distribution of the refractive index.

4. The device according to claim 3, wherein the electromagnetic wave-projecting means for projecting the electromagnetic wave projects an electromagnetic wave with a wavelength near an edge of the wavelength band and the detecting means measures the intensity of emitted electromagnetic wave.

5. The device according to claim 3, wherein the periodic structure has a defect in the regular arrangement of the vacant portion and the solid portion to provide an electromagnetic wave-transmissive wavelength range in the wavelength band where the electromagnetic wave propagation is forbidden, the electromagnetic wave-projecting means projects the electromagnetic wave in the electromagnetic wave-transmissive wavelength range to the periodic structure, and the detecting means measures the electromagnetic wave of the electromagnetic wave-transmissive wavelength range emitted from the periodic structure.

6. The device according to claim 1, wherein the device has additionally a temperature-controlling means for controlling the temperature of the periodic structure.

7. The device according to claim 1, wherein the device has additionally a polarization-controlling means for controlling polarization of the electromagnetic wave.

8. The device according to claim 1, wherein the electromagnetic wave projected to the periodic structure has a continuous wavelength component, and the detecting means measures the spectrum of the electromagnetic wave emitted from the periodic structure.

9. The device according to claim 1, wherein the electromagnetic wave is projected through a collimating means onto the periodic structure, and the detecting means measures the direction of transmission of the electromagnetic wave.

10. The device according to claim 1, wherein the device has additionally a first aligning means for aligning the electromagnetic wave emitted from the electromagnetic wave-projecting means to enter the periodic structure at a prescribed position at a prescribed angle, and a second aligning means for aligning the electromagnetic wave to reach the detecting means.

11. The device according to claim 1, wherein the solid portions of the structure are columnar, and the vacant portion is an interstice among the structure.

12. The device according to claim 1, wherein the solid portion is a continuous body and the vacant portion is constituted of holes penetrating the continuous body.

13. A device for detecting a target substance in a fluid, comprising
a flow path for passing a fluid containing the target substance,
a periodic structure placed at least a portion of the flow path and having a vacant portion for passing the fluid containing the target substance and a solid portion capable of transmitting an electromagnetic wave arranged regularly to form a periodic distribution of a refractive index for the electromagnetic wave,
an electromagnetic wave-projecting means for projecting the electromagnetic wave to the periodic structure, and
a detecting means for measuring the magnetic wave emitted from the periodic structure to detect a change in the periodic distribution of a refractive index.

14. The device according to claim 13, wherein the periodic structure has periodic distribution of the refractive index in a direction perpendicular to the flow path, and the electromagnetic wave is projected in the direction.

15. The device according to claim 13, wherein the periodic structure has periodic distribution of the refractive index in a direction parallel to the flow path, and the electromagnetic wave is projected in the direction.

16. The device according to claim 13, wherein the periodic structure has columnar solid portions regularly placed two-dimensionally with an interspace, and the plane of the periodic structure is parallel to the flow path.

17. The device according to claim 13, wherein the periodic structure is a two-dimensional periodic structure which has a continuous solid body and holes regularly placed and penetrating the continuous body, and the holes are parallel to the flow path.

18. A device for detecting plural target substances in a fluid, comprising
a flow path for passing a fluid containing the target substances;
plural periodic structures each of which is placed at least a portion of the flow path and has a vacant portion for passing the fluid containing the target substances and a solid portion capable of transmitting an electromagnetic wave arranged regularly to form a periodic distribution of a refractive index for the electromagnetic wave,
an electromagnetic wave-projecting means for projecting the electromagnetic wave to the periodic structures, and
a detecting means for measuring the magnetic wave emitted from the periodic structures to detect a change in the periodic distribution of the refractive index.

19. The device according to claim 18, wherein the periodic structure forbids transmission of a specific wavelength band of the electromagnetic wave defined by the periodic distribution of the refractive index.

20. The device according to claim 18, wherein the periodic structures have respectively a different trapping substance distributed on the surface of the solid portion and capable of bonding to one of the target substances; and the detecting means detects the respective changes in the periodic distribution of the refractive indexes caused by the target substance and the trapping substance.

21. The device according to claim 19, wherein the plural periodic structures are placed in series along the flow path, and plural electromagnetic wave-projecting means for projecting the electromagnetic wave in the direction perpendicular to the flow path to the respective periodic structure, and plural detecting parts for detecting the magnetic waves emitted from the periodic structures are provided.

22. The device according to claim 21, wherein the periodic structures have the same construction in the same dimension, and the electromagnetic wave-projecting means project respectively an electromagnetic wave of the wavelength near the band edge of the wavelength band while the trapping substances capable of bonding to target substances are distributed on the surface of the solid portion.

23. The device according to claim 22, wherein the periodic structures have respectively a nearly the same band edge wavelength of the wavelength band in a state that the trapping substances capable of bonding to target substances are distributed on the surface of the solid portion, and the electromagnetic wave-projecting means project respectively an electromagnetic wave of the band edge wavelength.

24. The device according to claim 18, wherein the electromagnetic waves projected to the periodic structures are generated from one and the same electromagnetic wave source, and the produced electromagnetic wave is split and projected to the periodic structures.

25. The device according to claim 19, wherein the plural periodic structures are placed in series along the flow path, and plural electromagnetic wave-projecting means for projecting the electromagnetic wave in the direction parallel to the flow path to the respective periodic structure, and plural detecting parts for detecting the magnetic waves emitted from the periodic structures are provided.

26. The device according to claim 25, wherein the periodic structures have the wavelengths not overlapping with each other, and the projected electromagnetic wave is emitted from a wavelength-variable electromagnetic wave source including the band edge wavelengths of the wavelength bands.

27. The device according to claim 19, wherein the plural periodic structures are placed in parallel in the flow path, and an electromagnetic wave-projecting means for projecting the electromagnetic wave in the parallel placement to the respective periodic structures, and a detecting unit for detecting the magnetic wave transmitted and emitted from the periodic structures are provided.

28. The device according to claim 27, wherein the periodic structures have the wavelengths not overlapping with each other, and the projected electromagnetic wave is emitted from a wavelength-variable electromagnetic wave source including the band edge wavelengths of the wavelength bands.

29. A device for detecting a target substance in a fluid, comprising
an optical fiber having plural holes for passing the fluid containing the target substance and a solid portion capable of transmitting an electromagnetic wave to form a refractive index distribution in the radius direction,
an electromagnetic wave-introducing means for introducing the electromagnetic wave to the optical fiber, and
a detecting means for measuring the magnetic wave emitted from the optical fiber in the radius direction to detect a change in a refractive index.

30. The device according to claim 29, wherein the optical fiber is a photonic crystal fiber having plural holes arranged regularly and having periodic structure of the refractive index in the fiber radius direction.

31. The device according to claim 30, wherein the trapping substance for bonding selectively to the target substance is disposed on the surface of the holes.
